(19) United States
(12) Patent Application Publication
(10) Pub. No.: US 2026/0335229 A1
(43) Pub. Date: Oct. 29, 2026

(54) NOVEL COMPOSITIONS

(71) Applicant: HELM PHARMACEUTICALS GMBH, Hamburg (DE)

(72) Inventors: Jan JEZEK, Saffron Walden (GB); David GERRING, Saffron Walden (GB); Joshua CREMIN, Saffron Walden (GB); Jorge PINTO, Saffron Walden (GB)

(21) Appl. No.: 19/282,648

(22) Filed: Jul. 28, 2025

Related U.S. Application Data
(63) Continuation of application No. PCT/EP2025/061742, filed on Apr. 29, 2025.

(51) Int. Cl. A61K 9/08; A61K 38/14; A61K 47/02; A61K 47/10; A61K 47/12; A61K 47/18; A61K 47/22; A61K 47/26

(52) U.S. Cl. CPC A61K 9/08; A61K 38/14; A61K 47/02; A61K 47/10; A61K 47/12; A61K 47/183; A61K 47/22; A61K 47/26

(57) ABSTRACT

The invention relates to storage stable compositions of dalbavancin. In particular, the storage stable composition is an aqueous liquid solution comprising: dalbavancin or a pharmaceutically acceptable salt thereof; a neutral amino acid or a pharmaceutically acceptable salt thereof; and a non-ionic surfactant, wherein the pH of the composition is in the range of about 3.0 to about 7.0.

### FIELD OF INVENTION

The present invention relates to storage stable aqueous liquid solution compositions of dalbavancin, in particular, compositions which are diluted prior to administration, and which are non-toxic and have improved handling.

### BACKGROUND

Dalbavancin is a second-generation lipoglycopeptide antibiotic, which is produced via a semi-synthetic process in which the precursor glycopeptide complex A-40926, isolated from the actinomycete genus Nonomuraea, is selectively esterified, amidated and saponified. Dalbavancin is typically used in the form of its hydrochloride salt, which has a molecular weight of 1816.71 Da.

Dalbavancin has strong antibiotic activity against a number of Gram-positive bacteria including Staphylococcus aureus, Streptococcus pyogenes, Streptococcus agalactiae, Streptococcus anginosus, Streptococcus intermedius, and Streptococcus constellatus. In particular, dalbavancin is one of the most widely used antibiotics in the treatment of methicillin-resistant Staphylococcus aureus (MRSA). Clinically, dalbavancin is particularly used to treat acute bacterial skin and skin structure infections (ABSSSI), in particular when said infections are caused by the aforementioned Gram-positive bacteria, including MRSA.

Mechanistically, dalbavancin binds to the C-terminal lysyl-D-alanyl-D-alanine sub-unit of expanding peptidoglycan chains, resulting in the prevention of transpeptidation of said chains. Consequently peptidoglycan elongation, and therefore, in embodiments, bacterial cell wall formation and cell reproduction are inhibited.

Dalbavancin is typically administered intravenously, and accordingly the present invention disclosed herein is primarily concerned with storage stable aqueous liquid solution compositions of dalbavancin which, following dilution, are suitable for intravenous administration.

The existing commercial dalbavancin product Xydalba®/Dalvance® is a product for intravenous use that is lyophilised. This product is presented as a lyophilised powder which must be reconstituted and subsequently diluted into an intravenous (IV) solution bag prior to administration. These requirements mean that this product is somewhat inconvenient to use.

Xydalba®/Dalvance® is formulated in a single-use vial and contains 500 mg dalbavancin hydrochloride, 129 mg lactose monohydrate, 129 mg mannitol, and hydrochloric acid and/or sodium hydroxide (for pH adjustment). This formulation must be reconstituted with 25 ml of either sterile water for injection, USP, or 5% dextrose for injection. Following reconstitution, the pH of the resultant solution is between 5.0-7.0.

In view of the above-described lyophilised product, there is a need for storage stable aqueous liquid solution concentrated compositions of dalbavancin, which simply require dilution into an IV solution bag or bottle prior to use (i.e. intravenous administration). Relative to the existing lyophilised products, the benefit of such a concentrated liquid composition of dalbavancin would be that such a formulation removes the need for aseptic reconstitution, resulting in higher degree of patient safety and greater convenience for the healthcare professional. It would also offer greater dosing flexibility to address different dosing regimens.

The present invention addresses the need for storage stable and non-toxic dalbavancin compositions, that are simply diluted prior to administration, thereby providing greater dosing flexibility and improved handling.

The following documents relate to compositions comprising dalbavancin:
WO2018/096556A1 (GUFIC BIOSCIENCES LIMITED) discloses a lyophilized pharmaceutical composition comprising (a) dalbavancin hydrochloride or its pharmaceutically acceptable salt and (b) a pharmaceutically acceptable amount of an amino acid as a stabilizer, wherein the ratio of dalbavancin to stabilizer is 1:0.8 to 1:1 by weight.
U.S. Pat. No. 7,119,061B2 (VICURON PHARMACEUTICALS, INC.) discloses methods and compositions for treatment of bacterial infections, including administration of dalbavancin formulations.

The following documents primarily relate to compositions comprising an alternative lipoglycopeptide antibiotic, vancomycin:
WO2016/071495A1 (XELLIA PHARMACEUTICALS) discloses solution comprising a glycopeptide antibiotic, for example vancomycin, and an amino acid or amino acid derivative, such as N-acetyl-glycine or N-acetyl-D-alanine.
US2022/0133845A1 (SOMERSET THERAPEUTICS, LLC) discloses formulations and compositions comprising one or more glycopeptide antibiotics or a pharmaceutically acceptable salt thereof for parenteral administration, for example a liquid formulation comprising vancomycin, N-acetyl-methionine and one or more pharmaceutically acceptable excipients.
U.S. Pat. No. 10,471,149B2 (INSMED INCORPORATED) discloses stabilized lipid-based glycopeptide antibiotic compositions and a process for producing the same.
US2022/0040309A1 (CHILDREN'S MEDICAL CENTER CORPORATION) discloses compositions and methods for delivery of therapeutic agents across a barrier, for example comprising a therapeutic agent, a permeation enhancer, and a matrix forming agent.
WO2020/185518A1 (EMPHASCIENCE, INC.) discloses compositions comprising a cyclic peptide, a solvent, and two or more hydroxyihydrocarbons, independently selected from (i) ethanol and glycerol or (ii) ethanol and mannitol.

### SUMMARY OF THE INVENTION

The present invention provides, inter alia, a storage stable aqueous liquid solution composition comprising:
(i) dalbavancin or a pharmaceutically acceptable salt thereof;
(ii) a neutral amino acid or a pharmaceutically acceptable salt thereof; and
(iii) a non-ionic surfactant;
wherein the pH of the composition is in the range of about 3.0 to about 7.0.

### DETAILED DESCRIPTION OF THE INVENTION

In the storage stable aqueous liquid solution compositions of the present invention, dalbavancin may be present in the form of a pharmaceutically acceptable salt. Examples of pharmaceutically acceptable salts include hydrochloride, citrate, formate, acetate, tartrate, sulphate, tosylate, nitrate, mesylate, napsylate, besylate, oxalate, maleate, phosphate, pamoate, fumarate, hippurate, succinate and carbonate. In embodiments, dalbavancin is present in the composition as dalbavancin i.e. as the free base. In embodiments, dalbavancin is present in the composition as dalbavancin hydrochloride. For the avoidance of doubt, any reference herein to “dalbavancin” is also intended to cover a pharmaceutically acceptable salt of dalbavancin, in particular the hydrochloride salt, unless stated otherwise.

In embodiments, the compositions of the present invention are concentrated solutions of dalbavancin (i.e. a composition concentrate), which require dilution into an IV solution bag or bottle prior to use. In embodiments, the compositions of the present invention contain dalbavancin at a concentrations which is higher than that required for administration, in particular intravenous administration. In embodiments, the compositions of the present invention are therefore typically diluted to the required concentration shortly before administration, e.g. intravenous administration. Prior to use, in embodiments, the composition is typically stored in a suitable container such as a sealed vial. In embodiments, the concentration of dalbavancin or a pharmaceutically acceptable salt thereof in the compositions of the invention is 5-50 mg/mL, for example 5-40 mg/mL, for example 10-40 mg/mL, for example 10-35 mg/mL, for example 10-30 mg/mL, for example 10-25 mg/mL, for example 15-25 mg/mL, or about 20 mg/mL. The aforementioned concentrations are based on dalbavancin and will be scaled accordingly when a salt such as the hydrochloride salt is used.

In embodiments, the compositions of the present invention further comprise a neutral amino acid. As used herein, a neutral amino acid is an amino acid the side chain of which does not contain an ionisable group which is significantly ionised (e.g. more than 5% especially more than 10% of the side chain have a negative or positive charge) at the pH of the composition. For example, the neutral amino acid does not contain a side chain with a pKa in the range 2.5-7.5 e.g. 3.0-7.0. Suitably, the neutral amino acid is selected from the group consisting of glycine, methionine, proline, alanine, valine, leucine, isoleucine, phenylalanine, tyrosine, tryptophan, serine, threonine, asparagine and glutamine, and pharmaceutically acceptable salts thereof, and combinations thereof. In a particularly suitable embodiment, the neutral amino acid is selected from the group consisting of glycine, methionine, proline, and pharmaceutically acceptable salts thereof, and combinations thereof. In embodiments, the neutral amino acid is glycine. In a further embodiment, the neutral amino acid is methionine. In a preferred embodiment, the neutral amino acid is proline. For the purposes of this specification, cysteine is not a neutral amino acid. If cysteine is included in the composition of the invention it is included as an antioxidant as described elsewhere herein.

In embodiments, suitable pharmaceutically acceptable salts of a neutral amino acid include those listed above as suitable pharmaceutically acceptable salts of dalbavancin. Suitable pharmaceutically acceptable salts of a neutral amino acid also include those formed with cations, for example sodium, potassium or calcium ions. Suitably the neutral amino acid is used not in the form of a pharmaceutically acceptable salt. Neutral amino acids may be employed in the L or D forms or mixtures thereof such as racemic mixtures. Suitably the L form is employed. In embodiments, the neutral amino acid is present in the form of the L isomer thereof. In particular, in a preferred embodiment, the neutral amino acid is L-proline.

In embodiments, the total concentration of the neutral amino acid (i.e. the one or more neutral amino acids) in the compositions of the invention is 50-500 mM, for example 50-450 mM, for example 50-400 mM, for example 100-400 mM, for example 100-350 mM, for example 100-300 mM. For example, the total concentration of the neutral amino acid (i.e. the one or more neutral amino acids) in the compositions of the invention is 100-250 mM, such as about 100 mM or such as about 150 mM, or such as about 200 mM, or such as about 225 mM, or such as about 250 mM.

In embodiments, the compositions of the present invention further comprise a non-ionic surfactant. In embodiments, the non-ionic surfactant is suitably selected from the group consisting of a polysorbate, an alkyl glycoside, an alkyl ether of polyethylene glycol, a block copolymer of polyethylene glycol and polypropylene glycol, and an alkylphenyl ether of polyethylene glycol, and combinations thereof.

In embodiments, there is a particularly suitable class of non-ionic surfactants, which is the polysorbate surfactants, i.e. polysorbates, (fatty acid esters of ethoxylated sorbitan), such as polysorbate 20, or polysorbate 80. Polysorbate 20 is a mono ester formed from lauric acid and polyoxyethylene (20) sorbitan in which the number 20 indicates the number of oxyethylene groups in the molecule. Polysorbate 80 is a mono ester formed from oleic acid and polyoxyethylene (20) sorbitan in which the number 20 indicates the number of oxyethylene groups in the molecule. Polysorbate 20 is known under a range of brand names including in particular Tween 20, and also Alkest TW 20. Polysorbate 80 is known under a range of brand names including in particular Tween 80, and also Alkest TW 80. Other suitable polysorbate surfactants, i.e. polysorbates, include polysorbate 40 and polysorbate 60. Therefore, in embodiments, the non-ionic surfactant is a polysorbate surfactant, such as polysorbate 20, polysorbate 40, polysorbate 60, or polysorbate 80. For example, In embodiments, the non-ionic surfactant is a polysorbate surfactant, such as polysorbate 20 or polysorbate 80. In one preferred embodiment, the non-ionic surfactant is polysorbate 80.

Another suitable class of non-ionic surfactants is the alkyl glycosides. Suitable alkyl glycosides include dodecyl maltoside, dodecyl glucoside, octyl glucoside, octyl maltoside, decyl glucoside, decyl maltoside, tridecyl glucoside, tridecyl maltoside, tetradecyl glucoside, tetradecyl maltoside, hexadecyl glucoside, hexadecyl maltoside, sucrose monooctanoate, sucrose mono decanoate, sucrose monododecanoate, sucrose monotridecanoate, sucrose monotetradecanoate and sucrose monohexadecanoate. A particularly suitable alkyl glycoside is dodecyl maltoside.

In embodiments, a further suitable class of non-ionic surfactants are the alkyl ethers of polyethylene glycol, such as those known under a brand name Brij, such as selected from polyethylene glycol (2) hexadecyl ether (Brij 52), polyethylene glycol (2) oleyl ether (Brij 93) and polyethylene glycol (2) dodecyl ether (Brij L4). Other suitable Brij surfactants include polyethylene glycol (4) lauryl ether (Brij 30), polyethylene glycol (10) lauryl ether (Brij 35), polyethylene glycol (20) hexadecyl ether (Brij 58) and polyethylene glycol (10) stearyl ether (Brij 78).

In embodiments, a suitable class of non-ionic surfactants are the block copolymers of polyethylene glycol and polypropylene glycol, also known as poloxamers. Suitable poloxamer surfactants include poloxamer 188, poloxamer 407, poloxamer 171 and poloxamer 185. Poloxamer surfactants are also known under the brand name Pluronics or the brand name Koliphors. For example, poloxamer 188 is marketed as Pluronic F-68. In embodiments, the non-ionic surfactant is a block copolymer of polyethylene glycol and polypropylene glycol, i.e. it is a poloxamer surfactant. For example, In embodiments, the non-ionic surfactant which is a block copolymer of polyethylene glycol and polypropylene glycol is selected from the group consisting of poloxamer 188, poloxamer 407, poloxamer 171, and poloxamer 185. In one particular embodiment, the non-ionic surfactant is poloxamer 188.

In embodiments, a suitable class of non-ionic surfactants are the alkylphenyl ethers of polyethylene glycol, such as 4-(1,1,3,3-tetramethylbutyl)phenyl-polyethylene glycol, also known under the brand name Triton X-100.

In embodiments, the total concentration of the non-ionic surfactant (i.e. the one or more non-ionic surfactants) in the compositions of the invention is about 0.5 mg/ml or more, for example about 1 mg/mL or more, for example about 2 mg/ml or more, for example about 2.5 mg/ml or more, for example about 4 mg/mL or more, for example about 5 mg/ml or more.

In embodiments, the total concentration of the non-ionic surfactant (i.e. the one or more non-ionic surfactants) in the compositions of the invention is about 30 mg/ml or less, for example about 25 mg/mL or less, for example about 20 mg/mL or less, for example about 18 mg/ml or less, for example about 16 mg/ml or less, for example about 15 mg/ml or less.

In embodiments, the total concentration of the non-ionic surfactant (i.e. the one or more non-ionic surfactants) in the compositions of the invention is about 0.5-30 mg/mL, for example about 0.5-25 mg/mL, for example about 1-20 mg/mL, for example about 2-20 mg/mL, for example 5-20 mg/ml or 2-18 mg/mL, for example 5-15 mg/mL, or 2-15 mg/mL. For example, In embodiments, the total concentration of the non-ionic surfactant (i.e. the one or more non-ionic surfactants) in the compositions of the invention is about 10 mg/mL, or about 15 mg/mL, or about 20 mg/mL.

Suitably, the pH of the storage stable aqueous liquid compositions according to the present invention is in the range of about of about 3.0 to about 7.0 (in particular, 3.0 to 7.0), for example about 3.5 to 7.0, for example about 3.5 to 6.5, for example about 3.5 to 6.0, for example about 4.0 to about 6.0, for example about 4.0 to about 5.5, for example about 4.0 to about 5.0, such as about 4.5 or about 5.0, or about 4.5 to 5.5, such as about 5.0 or about 5.5. In one particular embodiment, the pH of the composition of the present invention is about 4.5 to 5.5, such as about 5.0.

In embodiments, the storage stable aqueous liquid solution compositions of the present invention further comprise an additional salt at a total concentration of 50 mM or less, for example 40 mM or less, for example 30 mM or less, for example 20 mM or less, for example 10 mM or less, for example 5 mM or less, for example 2 mM or less. In particular, In embodiments, the compositions of the present invention comprise an additional salt at a total concentration of 0-50 mM, for example 0-40 mM, for example 0-30 mM, for example 0-20 mM, for example 0-10 mM, for example 0-5 mM, for example 0-2 mM. Alternatively, In embodiments, the compositions of the present invention comprise an additional salt at a total concentration of 0.1-50 mM, for example 0.1-40 mM, for example 0.1-30 mM, for example 0.1-20 mM, for example 0.1-10 mM, for example 0.1-5 mM, for example 0.1-2 mM. In a particularly preferred embodiment, the compositions of the present invention are substantially free of, for example are free of, an additional salt. As used herein, “an additional salt” refers to any salt other than a salt which is included in the composition as a result of a specific formulation excipient being included in the composition in the salt form. That is, any salt which is included in the composition of the present invention that is not e.g. a salt of dalbavancin and/or a salt of a neutral amino acid and/or a buffer salt. Suitably, the additional salt, that is present in the composition at a total concentration of 50 mM or less, or that the composition is substantially free of, is sodium chloride, sodium lactate, or combinations thereof. Suitably, an additional salt, that is present in the composition at a total concentration of 50 mM or less, or that the composition is substantially free of, is sodium chloride. Suitably an additional salt, that is present in the composition at a total concentration of 50 mM or less, or that the composition is substantially free of, is of sodium lactate.

Sources of sodium chloride in the formulation which are not added as an additional salt include chloride ions and sodium ions from pH adjustment using, respectively, hydrochloric acid and sodium hydroxide as well as chloride ions from dalbavancin hydrochloride when the chloride salt is used or chloride ions and sodium ions from salt forms of the neutral amino acids present in the composition. Suitably the total concentration of sodium chloride in the formulation from whatever source is 50 mM or less, for example 40 mM or less, for example 30 mM or less, for example 20 mM or less, for example 10 mM or less, for example 5 mM or less, for example 2 mM or less e.g. is 0-20 mM e.g. 2-20 mM e.g. 5-20 mM e.g. 10-20 mM or 0-10 mM e.g. 2-10 mM e.g. 5-10 mM.

Where present, such additional salts may contribute to the tonicity of the composition and act as a charged tonicity modifier.

In embodiments, the storage stable aqueous liquid solution compositions of the present invention are substantially free of, for example are free of, N-acetyl-D-alanine and/or N-acetyl-glycine and/or N-acetyl-methionine. For example, the storage stable aqueous liquid solution compositions of the present invention are substantially free of, for example are free of, N-acetyl-D-alanine and N-acetyl-glycine and N-acetyl-methionine.

“Substantially free of” as used herein means that the composition concentrate contains <0.1 mg/ml of the stated component if the component is a solid at 21 °C, or <0.01% (v/v) if the component is a liquid at 21 °C.

In embodiments, the compositions of the present invention may further comprise a buffer. The compositions of the present invention comprise a buffer, in particular a buffer with suitable buffering capacity in the pH range of about 3.0 to about 7.0, such as in the pH range of about 4.0 to about 6.0, such as in the pH range 4.5 to 5.5. Suitably, the composition of the present invention comprises a buffer selected from the group consisting of maleate, sulphite, aspartame, aspartate, glutamate, tartrate, gluconate, adenine, succinate, ascorbate, benzoate, phenylacetate, gallate, cytosine, p-aminobenzoic acid, sorbate, acetate, propionate, lactate, alginate, urate, 2-(N-morpholino) ethanesulphonic acid, bicarbonate, bis(2-hydroxyethyl)iminotris(hydroxymethyl) methane, N-(2-acetamido)-2-iminodiacetic acid, 2-[(2-amino-2-oxoethyl)amino]ethanesulphonic acid, piperazine-N,N′-bis(2-ethanesulphonic acid) (PIPES), phosphate, N,N-bis(2-hydroxyethyl)-2-aminoethanesulphonic acid, 3-[N,N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane, N tris(hydroxymethyl)glycine, N-tris(hydroxymethyl)methyl-3-aminopropanesulphonic acid, citrate and salts thereof, and combinations thereof. In one particular embodiment, the composition of the present invention comprises a buffer selected from the group consisting of maleate, succinate, acetate and citrate. In one particularly suitable embodiment, the compositions of the present invention comprise acetate buffer.

It will be understood that the skilled person will understand that buffers are present in solution at the target pH in an equilibrium between a protonated and deprotonated form. Hence the reference to “citrate” or “lactate” etc. as buffer will be understood to mean a mixture of that ion and the corresponding acid as buffer in a ratio according to the target pH. In this context, “target pH” means the pH at which the composition of the present invention is intended to be buffered. At a pH below the pKa of the ionisable group the protonated form predominates. At a pH above the pKa of the ionisable group the deprotonated form predominates. A buffer is most effective at controlling (i.e. buffering) pH at pH values within 2 pH units or more suitably within around 1 pH unit of the pKa of the or an ionisable group of the buffer. When a buffer is included in a composition of the present invention, the buffer acid (e.g. citric acid, lactic acid etc) may be added and the pH raised to the desired pH by addition of a base such as sodium hydroxide.

When a buffer is included in a composition of the present invention, the total concentration of the buffer (i.e. the one or more buffers) is suitably in the range about 1-20 mM, for example about 2-20 mM, for example about 2-18 mM, for example about 4-16 mM, for example about 5-15 mM, for example about 5-12 mM, for example about 5-10 mM. Suitably, the total concentration of the buffer (i.e. the one or more buffers) is about 5 mM, about 6 mM, about 7 mM, about 8 mM, about 9 mM, or about 10 mM.

In embodiments, the compositions of the present invention may further comprise a polyol. Suitably, the polyol may function as an uncharged tonicity modifier. For example, In embodiments, the polyol is present as a stabilizer and, although the polyol contributes to tonicity, a further additional tonicity modifier (i.e. in addition to the polyol) is included in the composition of the present invention. In an alternative embodiment, the polyol is present as a stabilizer and no further additional tonicity modifier (i.e. in addition to the polyol) is included in the composition of the present invention. It would be understood by the skilled person that, as the storage stable aqueous liquid compositions of the present invention are diluted prior to use, any tonicity modifier, for example an uncharged tonicity modifier such as a polyol, would be included in said compositions at a concentration which results in the diluted compositions having the desired tonicity, e.g. are isotonic, or slightly hypertonic.

In embodiments, the polyol is selected from the group consisting of polyethylene glycol (PEG) 300, PEG400 propylene glycol, sucrose, glycerol, trehalose, lactose, glucose, sorbitol and mannitol and combinations thereof. For example, the polyol is suitably selected from the group consisting of propylene glycol, glycerol, mannitol, and glucose, and combinations thereof. In particular, In embodiments, the polyol is selected from the group consisting of glucose and propylene glycol. In particular, In embodiments, the polyol is glucose. In particular, In embodiments, the polyol is propylene glycol. In embodiments, the polyol is not cyclodextrin.

When a polyol is included in the compositions of the present invention, the total concentration of the polyol (i.e. the one or more polyols) is suitably in the range about 10-500 mM, for example about 10-450 mM, for example about 10-400 mM, for example about 20-400 mM, for example about 20-350 mM, for example about 25-350 mM, for example 50-350 mM, for example 50-300 mM.

As noted above, the compositions of the present invention may further comprise a tonicity modifier, particularly an uncharged tonicity modifier. Examples of uncharged tonicity modifiers include the polyols referred to above, such as PEG300, PEG400 propylene glycol, sucrose, glycerol, trehalose, lactose, glucose, sorbitol and mannitol and combinations thereof.

When included in a composition of the present invention, an uncharged tonicity modifier is typically employed in the composition at a concentration of 50-1000 mM, for example 200-500 mM, such as about 300 mM.

As noted above, where an additional salt is present this may act as a charged tonicity modifier and thus contribute to tonicity.

In embodiments, the compositions of the present invention may further comprise an antioxidant. Suitably, the antioxidant is selected from the group consisting of monothioglycerol, butylated hydroxyanisole, glutathione (reduced), ascorbate, and cysteine. Monothioglycerol is also known as 1-thioglycerol, α-monothioglycerol, α-thioglycerol and 3-mercapto-1,2-propanediol. When included in the compositions of the present invention, the antioxidant is suitably present at a concentration of about 1 to about 100 mM, for example about 2 to about 80 mM, for example about 5 to about 50 mM, for example about 10 to about 50 mM, for example about 25 to about 50 mM. It should be noted that methionine, which is identified above as a suitable neutral amino acid, has antioxidant properties. Therefore, in embodiments, if methionine is present in the compositions of the present invention as a neutral amino acid, it may also serve as an antioxidant. When methionine is present as an antioxidant in the compositions of the present invention, the concentration of methionine present as an antioxidant is included in the total concentration of neutral amino acid referred to above.

In embodiments, the compositions of the present invention may further comprise a preservative, such as a phenolic or benzylic preservative. The preservative is suitably selected from the group consisting of phenol, m-cresol, chlorocresol, benzyl alcohol, propyl paraben and methyl paraben, in particular phenol, m-cresol and benzyl alcohol, and mixtures thereof. When included in the compositions of the present invention, the preservative is suitably present at a concentration of about 10-100 mM, for example 20-80 mM, such as 25-50 mM. The optimal concentration of the preservative in the composition is selected to ensure the composition passes the Pharmacopoeia Antimicrobial Effectiveness Test (USP <51>, Vol. 32).

The total ionic strength of the storage stable aqueous liquid solution compositions of the present invention, excluding the contribution of dalbavancin or the pharmaceutically acceptable salt thereof, is suitably less than 100 mM, for example less than 80 mM, for example less than 75 mM, for example less than 60 mM, for example less than 50 mM, for example less than 40 mM, less than 35 mM, less than 30 mM, less than 25 mM or less than 20 mM. The term “total ionic strength” is used herein as the following function of the concentration of all ions in a solution:

$$I = {\sum }_{X = 1}^{n} {c}_{x} {z}_{x}^{2} / 2$$

where cₓ is molar concentration of ion x (mol L⁻³), zₓ is the net charge of ion cₓ. The sum covers all ions (n) present in the solution excluding the contribution of dalbavancin or the pharmaceutically acceptable salt thereof. It will be understood that neutral amino acids have a net charge of zero in the compositions of the invention and do not thus contribute to the total ionic strength. In any event, the contribution of any neutral amino acid, or the pharmaceutically acceptable salt thereof, is not included.

The term “aqueous solution”, as used herein, refers to a solution in water, preferably distilled water, deionized water, water for injection, sterile water for injection or bacteriostatic water for injection. The compositions of the present invention are aqueous liquid solution compositions and therefore, in embodiments, comprise at least 75% (v/v) of water, such as at least 80% (v/v), at least 85% (v/V), at least 90% (v/V), or at least 95% (v/v) of water, e.g. sterile water for injection or bacteriostatic water for injection.

In embodiments, the present invention provides a storage stable aqueous liquid solution composition comprising:
dalbavancin or a pharmaceutically acceptable salt thereof, at a concentration of 5-50 mg/mL, for example 10-30 mg/mL, or about 20 mg/mL;
a neutral amino acid or a pharmaceutically acceptable salt thereof, at a concentration of 50-500 mM, for example 50-400 mM, for example 100-400 mM, for example 100-300 mM;
a non-ionic surfactant, at a concentration of 1-20 mg/mL, for example 2-20 mg/mL, for example 5-20 mg/mL, for example 5-15 mg/ml;
   wherein the pH of the composition is in the range of about 3.0 to about 7.0, for example in the range of about 4.0 to about 6.0, such as about 5.0.

In embodiments, the present invention provides a storage stable aqueous liquid solution composition comprising:
dalbavancin or a pharmaceutically acceptable salt thereof, e.g. dalbavancin hydrochloride, at a concentration of 5-50 mg/mL, for example 10-30 mg/mL, or about 20 mg/mL;
a neutral amino acid selected from the group consisting of glycine, methionine, proline, alanine, valine, leucine, isoleucine, phenylalanine, tyrosine, tryptophan, serine, threonine, asparagine, glutamine, and pharmaceutically acceptable salts thereof, and combinations thereof, at a concentration of 50-500 mM, for example 50-400 mM, for example 100-400 mM, for example 100-300 mM;
a non-ionic surfactant, selected from the group consisting of an alkyl glycoside, a polysorbate, an alkyl ether of polyethylene glycol, a block copolymer of polyethylene glycol and polypropylene glycol, and an alkylphenyl ether of polyethylene glycol, and combinations thereof, at a concentration of 1-20 mg/mL, for example 2-20 mg/mL, for example 5-20 mg/mL, for example 5-15 mg/ml;
   wherein the pH of the composition is in the range of about 3.0 to about 7.0, for example in the range of about 4.0 to about 6.0, such as about 5.0.

In embodiments, the present invention provides a storage stable aqueous liquid solution composition comprising:
dalbavancin or a pharmaceutically acceptable salt thereof, e.g. dalbavancin hydrochloride, at a concentration of 5-50 mg/mL, for example 10-30 mg/mL, or about 20 mg/ml;
a neutral amino acid selected from the group consisting of glycine, methionine, and proline, and pharmaceutically acceptable salts thereof, and combinations thereof, at a concentration of 50-500 mM, for example 50-400 mM, for example 100-400 mM, for example 100-300 mM;
a non-ionic surfactant, which is a polysorbate surfactant, such as polysorbate 20, polysorbate 40, polysorbate 60, or polysorbate 80, and combinations thereof, at a concentration of 1-20 mg/mL, for example 2-20 mg/mL, for example 5-20 mg/mL, for example 5-15 mg/mL;
   wherein the pH of the composition is in the range of about 3.0 to about 7.0, for example in the range of about 4.0 to about 6.0, such as about 5.0.

In embodiments, the present invention provides a storage stable aqueous liquid solution composition comprising:
dalbavancin or a pharmaceutically acceptable salt thereof, e.g. dalbavancin hydrochloride, at a concentration of 5-50 mg/mL, for example 10-30 mg/mL, or about 20 mg/ml;
a neutral amino acid selected from the group consisting of glycine and proline, and pharmaceutically acceptable salts thereof, and combinations thereof, at a concentration of 50-500 mM, for example 50-400 mM, for example 100-400 mM, for example 100-300 mM;
a non-ionic surfactant, which is polysorbate 20 or polysorbate 80, or combinations thereof, at a concentration of 1-20 mg/mL, for example 2-20 mg/mL, for example 5-20 mg/mL, for example 5-15 mg/ml;
   wherein the pH of the composition is in the range of about 3.0 to about 7.0, for example in the range of about 4.0 to about 6.0, such as about 5.0.

In embodiments, the present invention provides a storage stable aqueous liquid solution composition comprising:
dalbavancin or a pharmaceutically acceptable salt thereof, e.g. dalbavancin hydrochloride, at a concentration of 10-30 mg/mL, or about 20 mg/ml;
a neutral amino acid selected from the group consisting of glycine and proline, and pharmaceutically acceptable salts thereof, and combinations thereof, at a concentration of 100-400 mM, for example 100-300 mM;
a non-ionic surfactant, which is polysorbate 20 or polysorbate 80, or combinations thereof, at a concentration of 5-20 mg/mL, for example 5-15 mg/mL;
   wherein the pH of the composition is in the range of about 4.0 to about 6.0, such as about 5.0.

In embodiments, the present invention provides a storage stable aqueous liquid solution composition comprising:
dalbavancin or a pharmaceutically acceptable salt thereof, e.g. dalbavancin hydrochloride, at a concentration of 10-30 mg/mL, or about 20 mg/ml;
a neutral amino acid, which is glycine at a concentration of 100-400 mM, for example 100-300 mM;
a non-ionic surfactant, which is polysorbate 80, at a concentration of 5-20 mg/mL, for example 5-15 mg/ml;
   wherein the pH of the composition is in the range of about 4.0 to about 6.0, such as about 5.0.

In embodiments, the present invention provides a storage stable aqueous liquid solution composition comprising:
dalbavancin or a pharmaceutically acceptable salt thereof, e.g. dalbavancin hydrochloride, at a concentration of 10-30 mg/mL, or about 20 mg/ml;
a neutral amino acid, which is glycine at a concentration of 100-300 mM;
a non-ionic surfactant, which is polysorbate 80, at a concentration of 5-15 mg/ml;
   wherein the pH of the composition is in the range of about 4.0 to about 6.0, such as about 5.0.

In embodiments, the present invention provides a storage stable aqueous liquid solution composition comprising:
dalbavancin or a pharmaceutically acceptable salt thereof, e.g. dalbavancin hydrochloride, at a concentration of 10-30 mg/mL, or about 20 mg/ml;
a neutral amino acid, which is proline at a concentration of 100-400 mM, for example 100-300 mM;
a non-ionic surfactant, which is polysorbate 80, at a concentration of 5-20 mg/mL, for example 5-15 mg/ml;
   wherein the pH of the composition is in the range of about 4.0 to about 6.0, such as about 5.0.

In embodiments, the present invention provides a storage stable aqueous liquid solution composition comprising:
dalbavancin or a pharmaceutically acceptable salt thereof, e.g. dalbavancin hydrochloride, at a concentration of 10-30 mg/mL, or about 20 mg/mL;
a neutral amino acid, which is proline at a concentration of 100-300 mM;
a non-ionic surfactant, which is polysorbate 80, at a concentration of 5-15 mg/ml;
   wherein the pH of the composition is in the range of about 4.0 to about 6.0, such as about 5.0.

In embodiments, the present invention provides a storage stable aqueous liquid solution composition comprising:
dalbavancin or a pharmaceutically acceptable salt thereof, e.g. dalbavancin hydrochloride, at a concentration of 10-30 mg/mL, or about 20 mg/ml;
a neutral amino acid, which is L-proline at a concentration of 100-400 mM, for example 100-300 mM;
a non-ionic surfactant, which is polysorbate 80, at a concentration of 5-20 mg/mL, for example 5-15 mg/mL;
   wherein the pH of the composition is in the range of about 4.0 to about 6.0, such as about 5.0.

In embodiments, the present invention provides a storage stable aqueous liquid solution composition comprising:
dalbavancin or a pharmaceutically acceptable salt thereof, e.g. dalbavancin hydrochloride, at a concentration of 10-30 mg/mL, or about 20 mg/mL;
a neutral amino acid, which is L-proline at a concentration of 100-300 mM;
a non-ionic surfactant, which is polysorbate 80, at a concentration of 5-15 mg/ml;
   wherein the pH of the composition is in the range of about 4.0 to about 6.0, such as about 5.0.

In embodiments, the present invention provides a storage stable aqueous liquid solution composition comprising:
dalbavancin, as dalbavancin hydrochloride, at a concentration of about 20 mg/ml;
L-proline at a concentration of about 225 mM
polysorbate 80 at a concentration of about 10 mg/mL;
acetate buffer at a concentration of about 10 mM;
   wherein the pH of the composition is about 5.0.

In embodiments, the present invention provides a storage stable aqueous liquid solution composition comprising:
dalbavancin, as dalbavancin hydrochloride, at a concentration of about 20 mg/mL;
L-proline at a concentration of about 225 mM
polysorbate 80 at a concentration of about 20 mg/ml;
acetate buffer at a concentration of about 10 mM;
   wherein the pH of the composition is about 5.0.

In embodiments, the present invention provides a storage stable aqueous liquid solution composition consisting of, or consisting essentially of:
dalbavancin or a pharmaceutically acceptable salt thereof, at a concentration of 5-50 mg/mL, for example 10-30 mg/mL, or about 20 mg/mL;
a neutral amino acid or a pharmaceutically acceptable salt thereof, at a concentration of 50-500 mM, for example 50-400 mM, for example 100-400 mM, for example 100-300 mM;
a non-ionic surfactant, at a concentration of 1-20 mg/mL, for example 2-20 mg/mL, for example 5-20 mg/mL, for example 5-15 mg/ml;
water, e.g. water for injection;
optionally a buffer;
optionally a polyol;
optionally an antioxidant;
optionally a preservative;
   wherein the pH of the composition is in the range of about 3.0 to about 7.0, for example in the range of about 4.0 to about 6.0, such as about 5.0.

In embodiments, the present invention provides a storage stable aqueous liquid solution composition consisting of, or consisting essentially of:
dalbavancin or a pharmaceutically acceptable salt thereof, e.g. dalbavancin hydrochloride, at a concentration of 5-50 mg/mL, for example 10-30 mg/mL, or about 20 mg/mL;
a neutral amino acid selected from the group consisting of glycine, methionine, proline, alanine, valine, leucine, isoleucine, phenylalanine, tyrosine, tryptophan, serine, threonine, asparagine, glutamine, and pharmaceutically acceptable salts thereof, and combinations thereof, at a concentration of 50-500 mM, for example 50-400 mM, for example 100-400 mM, for example 100-300 mM;
a non-ionic surfactant, selected from the group consisting of an alkyl glycoside, a polysorbate, an alkyl ether of polyethylene glycol, a block copolymer of polyethylene glycol and polypropylene glycol, and an alkylphenyl ether of polyethylene glycol, and combinations thereof, at a concentration of 1-20 mg/mL, for example 2-20 mg/mL, for example 5-20 mg/mL, for example 5-15 mg/ml;
water, e.g. water for injection;
optionally a buffer;
optionally a polyol;
optionally an antioxidant;
optionally a preservative;
   wherein the pH of the composition is in the range of about 3.0 to about 7.0, for example in the range of about 4.0 to about 6.0, such as about 5.0.

In embodiments, the present invention provides a storage stable aqueous liquid solution composition consisting of, or consisting essentially of:
dalbavancin or a pharmaceutically acceptable salt thereof, e.g. dalbavancin hydrochloride, at a concentration of 5-50 mg/mL, for example 10-30 mg/mL, or about 20 mg/ml;
a neutral amino acid selected from the group consisting of glycine, methionine, and proline, and pharmaceutically acceptable salts thereof, and combinations thereof, at a concentration of 50-500 mM, for example 50-400 mM, for example 100-400 mM, for example 100-300 mM;
a non-ionic surfactant, which is a polysorbate surfactant, such as polysorbate 20, polysorbate 40, polysorbate 60, or polysorbate 80, and combinations thereof, at a concentration of 1-20 mg/mL, for example 2-20 mg/mL, for example 5-20 mg/mL, for example 5-15 mg/mL;
water, e.g. water for injection;
optionally a buffer;
optionally a polyol;
optionally an antioxidant;
optionally a preservative;
   wherein the pH of the composition is in the range of about 3.0 to about 7.0, for example in the range of about 4.0 to about 6.0, such as about 5.0.

In embodiments, the present invention provides a storage stable aqueous liquid solution composition consisting of, or consisting essentially of:
dalbavancin or a pharmaceutically acceptable salt thereof, e.g. dalbavancin hydrochloride, at a concentration of 5-50 mg/mL, for example 10-30 mg/mL, or about 20 mg/mL;
a neutral amino acid selected from the group consisting of glycine and proline, and pharmaceutically acceptable salts thereof, and combinations thereof, at a concentration of 50-500 mM, for example 50-400 mM, for example 100-400 mM, for example 100-300 mM;
a non-ionic surfactant, which is polysorbate 20 or polysorbate 80, or combinations thereof, at a concentration of 1-20 mg/mL, for example 2-20 mg/mL, for example 5-20 mg/mL, for example 5-15 mg/mL;
water, e.g. water for injection;
optionally a buffer;
optionally a polyol;
optionally an antioxidant;
optionally a preservative;
   wherein the pH of the composition is in the range of about 3.0 to about 7.0, for example in the range of about 4.0 to about 6.0, such as about 5.0.

In embodiments, the present invention provides a storage stable aqueous liquid solution composition consisting of, or consisting essentially of:
dalbavancin or a pharmaceutically acceptable salt thereof, e.g. dalbavancin hydrochloride, at a concentration of 10-30 mg/mL, or about 20 mg/ml;
a neutral amino acid selected from the group consisting of glycine and proline, and pharmaceutically acceptable salts thereof, and combinations thereof, at a concentration of 100-400 mM, for example 100-300 mM;
a non-ionic surfactant, which is polysorbate 20 or polysorbate 80, or combinations thereof, at a concentration of 5-20 mg/mL, for example 5-15 mg/ml;
water e.g. water for injection;
optionally a buffer;
optionally a polyol;
optionally an antioxidant;
optionally a preservative;
   wherein the pH of the composition is in the range of about 4.0 to about 6.0, such as about 5.0.

In embodiments, the present invention provides a storage stable aqueous liquid solution composition consisting of, or consisting essentially of:
dalbavancin or a pharmaceutically acceptable salt thereof, e.g. dalbavancin hydrochloride, at a concentration of 10-30 mg/mL, or about 20 mg/ml;
a neutral amino acid, which is glycine at a concentration of 100-400 mM, for example 100-300 mM;
a non-ionic surfactant, which is polysorbate 80, at a concentration of 5-20 mg/mL, for example 5-15 mg/mL;
water, e.g. water for injection;
optionally a buffer;
optionally a polyol;
optionally an antioxidant;
optionally a preservative;
   wherein the pH of the composition is in the range of about 4.0 to about 6.0, such as about 5.0.

In embodiments, the present invention provides a storage stable aqueous liquid solution composition consisting of, or consisting essentially of:
dalbavancin or a pharmaceutically acceptable salt thereof, e.g. dalbavancin hydrochloride, at a concentration of 10-30 mg/mL, or about 20 mg/mL;
a neutral amino acid, which is glycine at a concentration of 100-300 mM;
a non-ionic surfactant, which is polysorbate 80, at a concentration of 5-15 mg/mL;
water, e.g. water for injection;
optionally a buffer;
optionally a polyol;
optionally an antioxidant;
optionally a preservative;
   wherein the pH of the composition is in the range of about 4.0 to about 6.0, such as about 5.0.

In embodiments, the present invention provides a storage stable aqueous liquid solution composition consisting of, or consisting essentially of:
dalbavancin or a pharmaceutically acceptable salt thereof, e.g. dalbavancin hydrochloride, at a concentration of 10-30 mg/mL, or about 20 mg/mL;
a neutral amino acid, which is proline at a concentration of 100-400 mM, for example 100-300 mM;
a non-ionic surfactant, which is polysorbate 80, at a concentration of 5-20 mg/mL, for example 5-15 mg/ml;
water, e.g. water for injection;
optionally a buffer;
optionally a polyol;
optionally an antioxidant;
optionally a preservative;
   wherein the pH of the composition is in the range of about 4.0 to about 6.0, such as about 5.0.

In embodiments, the present invention provides a storage stable aqueous liquid solution composition consisting of, or consisting essentially of:
dalbavancin or a pharmaceutically acceptable salt thereof, e.g. dalbavancin hydrochloride, at a concentration of 10-30 mg/mL, or about 20 mg/ml;
a neutral amino acid, which is proline at a concentration of 100-300 mM;
a non-ionic surfactant, which is polysorbate 80, at a concentration of 5-15 mg/mL;
water, e.g. water for injection;
optionally a buffer;
optionally a polyol;
optionally an antioxidant;
optionally a preservative;
   wherein the pH of the composition is in the range of about 4.0 to about 6.0, such as about 5.0.

In embodiments, the present invention provides a storage stable aqueous liquid solution composition consisting of, or consisting essentially of:
dalbavancin or a pharmaceutically acceptable salt thereof, e.g. dalbavancin hydrochloride, at a concentration of 10-30 mg/mL, or about 20 mg/mL;
a neutral amino acid, which is L-proline at a concentration of 100-400 mM, for example 100-300 mM;
a non-ionic surfactant, which is polysorbate 80, at a concentration of 5-20 mg/mL, for example 5-15 mg/ml;
water, e.g. water for injection;
optionally a buffer;
optionally a polyol;
optionally an antioxidant;
optionally a preservative;
   wherein the pH of the composition is in the range of about 4.0 to about 6.0, such as about 5.0.

In embodiments, the present invention provides a storage stable aqueous liquid solution composition consisting of, or consisting essentially of:
dalbavancin or a pharmaceutically acceptable salt thereof, e.g. dalbavancin hydrochloride, at a concentration of 10-30 mg/mL, or about 20 mg/mL;
a neutral amino acid, which is L-proline at a concentration of 100-300 mM;
a non-ionic surfactant, which is polysorbate 80, at a concentration of 5-15 mg/ml;
water, e.g. water for injection;
optionally a buffer;
optionally a polyol;
optionally an antioxidant;
optionally a preservative;
   wherein the pH of the composition is in the range of about 4.0 to about 6.0, such as about 5.0.

In embodiments, the present invention provides a storage stable aqueous liquid solution composition consisting of, or consisting essentially of:
dalbavancin, as dalbavancin hydrochloride, at a concentration of about 20 mg/ml;
L-proline at a concentration of about 225 mM
polysorbate 80 at a concentration of about 10 mg/ml;
acetate buffer at a concentration of about 10 mM;
water, e.g. water for injection;
optionally a buffer;
optionally a polyol;
optionally an antioxidant;
optionally a preservative;
   wherein the pH of the composition is about 5.0.

In embodiments, the present invention provides a storage stable aqueous liquid solution composition consisting of, or consisting essentially of:
dalbavancin, as dalbavancin hydrochloride, at a concentration of about 20 mg/ml;
L-proline at a concentration of about 225 mM
polysorbate 80 at a concentration of about 20 mg/ml;
acetate buffer at a concentration of about 10 mM;
water, e.g. water for injection;
optionally a buffer;
optionally a polyol;
optionally an antioxidant;
optionally a preservative;
   wherein the pH of the composition is about 5.0.

As used herein when referring to the composition of the invention, the term “comprises”, and variations such as ‘comprises’ and ‘comprising’, means that the stated components are included in the compositions, but not to the exclusion of any other component or group of components other than those listed. As used herein when referring to the composition of the invention, the term “consists of” means that no further components are included in the composition other than those listed. As used herein when referring to the composition of the invention, the term “consists essentially of” means that specific further components can be present but said components do not materially affect the essential characteristics of the composition, and are typically present in a de minimis amount.

Methods for determining whether a formulation is storage stable would be known to a person skilled in the art. Stability testing provides evidence on how the quality of a drug substance or drug product varies with time under the influence of a variety of environmental factors such as temperature, humidity and light. Compositions of the invention may be considered storage stable if they meet the stability requirements for regulatory approval, for example as set out in the International Conference on Harmonization (ICH) Tripartite Guideline “Stability Testing of New Drug Substances and Products” which provides the stability testing requirements for a registration application within territories such as the European Union, Japan and the United States.

Compositions of the invention are storage stable, i.e. they remain as a clear solution (free of visible particles) following storage. Suitably the composition of the invention remains as a clear solution (e.g. as measured according to the Visual Assessment Method in the General Methods) following storage at 2-8 °C for an extended period of time, such as at least 6 months, at least 12 months, at least 18 months or at least 24 months. Visible particles may be suitably detected by methods known to those skilled in the art, such as the Visual Assessment Method or RP-HPLC methods described in the General Methods section herein, or in 2.9.20. European Pharmacopoeia Monograph (Particulate Contamination: Visible Particles).

Suitably the composition of the invention remains as a clear solution (e.g. as measured according to the Visual Assessment Method in the General Methods) following storage at 25 °C for at least 1 week, such as at least 2 weeks, at least 4 weeks or at least 8 weeks.

Suitably the composition of the invention comprises no more than 10% total impurities (by total weight of dalbavancin or pharmaceutically acceptable salt thereof in the composition) following storage at 2-8 °C for an extended period of time, such as at least 6 months, at least 12 months, at least 18 months or at least 24 months.

Suitably the composition of the invention comprises no more than 10% total impurities with (by total weight of dalbavancin or pharmaceutically acceptable salt thereof in the composition) following storage at 25 °C for at least 1 week, such as at least 2 weeks, at least 4 weeks or at least 8 weeks.

N-nitrosamines have been identified as a concerning impurity in pharmaceutical products because they are considered possible or probable human carcinogens. In particular, nitrosamine impurities are a problem associated with dalbavancin compositions currently known in the art. However, compositions of the present invention have a lower or reduced formation of nitrosamine impurities, in particular Nitrosamine Drug Substance-Related Impurities (NDSRIs), compared to compositions of dalbavancin known in the art. NDSRIs refer to a nitrosamine impurity or a class of nitrosamine impurities, whose structure is similar to the drug substance containing amine. The US Food and Drug Administration (FDA) have released guidance on Recommended Acceptable Intake Limits for Nitrosamine Drug Substance-Related Impurities (August 2023) (RAIL Guidance) which provides recommendations for NDSRI acceptable intake limits based on predicted carcinogenic potency categorization, as well as guidance on Control of Nitrosamine Impurities in Human Drugs (September 2024, Rev.2) (Nitrosamine Guidance), both of which are herein incorporated by reference. The current recommended Acceptable Intake (AI) limit for dalbavancin NDSRIs is 1500 ng/day.

NDSRIs relevant to compositions comprising dalbavancin or pharmaceutically acceptable salts thereof include, but are not limited to: N-nitroso-dalbavancin a0; N-nitroso-desmethyl-dalbavancin a0; N-nitroso-dalbavancin a1; N-nitroso-desmethyl-dalbavancin a1; N-nitroso-dalbavancin b0; N-nitroso-desmethyl-dalbavancin b0; N-nitroso-dalbavancin b1; N-nitroso-desmethyl-dalbavancin b1; and N-nitroso-desmethyl-dalbavancin b2.

Suitably the nitrosamine impurities in the composition of the invention are within acceptable levels. More suitably, the level of nitrosamine impurities in the composition of the invention does not increase (e.g. does not increase more than about 20%, 10%, 5% or 1%) compared to the level of nitrosamine impurities in the starting Active Pharmaceutical Ingredient (API). It will be understood that the “starting API” refers to dalbavancin or the pharmaceutically acceptable salt thereof used to prepare the composition of the invention.

Liquid chromatography methods may be used for detecting nitrosamine impurities, such as LCMS/MS. The level of nitrosamine impurities in the compositions may be expressed in parts per million (ppm) based on the weight of the drug substance containing amine. An Al limit can be converted into a ppm control limit based on a drug's maximum daily dose (MDD) as reflected in the drug labelling (ppm=Al (nanograms (ng))/MDD (milligrams)).

In addition, dalbavancin stability can also be tested via a microbiological bioassay that determines antibiotic potency. For a therapeutic product the method must comply with US Pharmacopeia method USP <81> (Microbial Assays) which is herein incorporated by reference. Briefly, dalbavancin samples are placed on agar plates seeded with suitable test microorganisms. The growth of the specific microorganisms inoculated into the agar is prevented in a circular area or “zone” around the sample. The extent of this inhibition is correlated to antibiotic potency and is reflective of dalbavancin stability. Compositions of the present invention have been shown to retain antibiotic potency, even after heat stress (data not shown).

The present invention also provides a container, containing a dose or a plurality of doses of the composition as described hereinabove. The container is suitably a vial e.g. a vial made of plastic or glass. Suitably a glass vial is a type 1 glass vial e.g. containing 80% silica and 10% boric oxide, with a small amount of sodium oxide and aluminium oxide. In one embodiment is provided a single-dose vial, in particular a glass vial, containing the composition as described hereinabove. In one embodiment is provided a multiple-dose vial, in particular a glass vial, containing the composition as described hereinabove. In embodiments, the fill volume of the vial is 10 mL, 20 mL, 25 mL, 40 mL, 50 mL, 75 mL or 100 mL, for example the fill volume of the vial is 25 mL.

The present invention also provides an injection device containing the diluted composition as described hereinabove. The injection device may be filled with a single dose of the composition as described herein.

The storage stable aqueous liquid solution composition of the invention is suitably diluted, in embodiments, prior to administration. Once diluted, the resulting solution is usually administered by intravenous infusion. A typical method for administration of a diluted composition of the invention involves transferring a volume of the storage stable aqueous liquid solution composition to a container which is suitably an intravenous (IV) solution bag or a bottle, where the volume of the diluted compositions to be administered is calculated according to the required dose and the volume of the container. Following thorough mixing of the diluted solution, the contents of the container can be administered to the patient.

When the container is an IV solution bag, it is suitably constructed from plastic such as PVC, polyolefin, polypropylene, a blend of PO and PP (polyolefin blend), polyurethane, or a material that is substantially free of PVC, plasticizers, adhesives and latex, such as DEHP (di(2-ethylhexyl) phthalate). When the container is a bottle, it can be made from plastic (as described above for the IV solution bag) or can be made from glass, e.g. it is a type 1 glass bottle. In embodiments, the volume of the IV solution bag or bottle is 50 ml to 1 L, e.g. 50 mL, 100 mL, 150 mL, 200 mL, 250 ml, 300 mL, 400 mL, 500 mL, 750 mL or 1 L.

The storage stable aqueous liquid solution composition of the invention can be diluted in a pharmaceutically acceptable aqueous diluent such as 0.9% (w/v) saline, 5% (w/v) glucose in water, or water (e.g. sterile water for injection or bacteriostatic water for injection).

Suitably, the storage stable aqueous liquid solution composition of the invention is diluted in a suitable aqueous diluent by mixing 1 volume part of the composition with 1-24 volume parts, such as 3-19 volume parts, such as 4-14 volume parts, (e.g. 9 volume parts) of the suitable aqueous diluent to achieve the described concentration of dalbavancin. The concentration of dalbavancin or pharmaceutically acceptable salt thereof in the resulting diluted composition is thus 2-24 times, such as 5-15 times (e.g. 10 times) lower than that in the storage stable aqueous liquid solution composition. In embodiments, the concentration of dalbavancin or pharmaceutically acceptable salt thereof in the diluted composition is 2-25%, such as 5-15% (e.g. 10%) of the concentration in the storage stable aqueous liquid solution composition. In embodiments, the diluted composition contains 0.5-10 mg/ml (e.g. 0.5-7.5 mg/ml, for example 1-5 mg/mL) dalbavancin (optionally in the form of a pharmaceutically acceptable salt e.g. the hydrochloride salt).

Therefore, in embodiments, the present invention provides a diluted composition, i.e. a diluted form of the storage stable aqueous liquid solution composition, wherein said storage stable aqueous liquid solution composition is diluted with an aqueous diluent, for example selected from 0.9% saline, 5% glucose in water, and water (e.g. sterile water for injection or bacteriostatic water for injection); and/or wherein the concentration of dalbavancin or pharmaceutically acceptable salt thereof in the diluted composition is 2-25%, for example 5-25%, for example 5-15%, such as about 10% of the concentration in the storage stable aqueous liquid solution composition prior to dilution.

In embodiments, the present invention provides a ready-to-administer composition comprising a storage stable liquid solution composition described herein, diluted with a diluent suitable for intravenous administration. A ready-to-use or ready-to-administer composition does not require reconstitution before use, but the composition can be further diluted if present as a concentrated solution, or directly administered. In embodiments, the ready-to-administer formulation is included at the required concentration and volume in a final administration device, such as an injection device (e.g. syringe) or infusion bag for administration to a patient.

Suitably the ready-to-administer or the diluted form of the composition of the invention is free of visible particles, i.e. it is clear and not opalescent as measured according to the procedures described herein.

Suitably the diluted composition of the invention is free of visible particles and is a colourless to slightly yellow solution. In embodiments, the diluted composition is isotonic. In another embodiment, the diluted composition is slightly hypertonic. Suitably, once prepared the diluted composition is stable for at least 24 hours, preferably for at least 48 hours at 2-8 °C and room light and at least 24 hours, preferably for at least 48 hours at 25 °C and room light.

Moreover, In embodiments, the present invention provides a method of preparing a solution i.e. a diluted composition, for administration, for example for intravenous administration, comprising diluting a storage stable aqueous liquid solution composition according to the invention with an aqueous diluent, for example wherein the aqueous diluent is selected from 0.9% saline, 5% glucose in water, and water (e.g. sterile water for injection or bacteriostatic water for injection). Suitably, the concentration of dalbavancin or pharmaceutically acceptable salt thereof in the diluted composition, prepared according to above-described method of the present invention, is about 2 to about 25%, for example about 5 to about 15%, such as about 10% of the concentration in the storage stable aqueous liquid solution composition prior to dilution; and/or wherein the concentration of dalbavancin or a pharmaceutically acceptable salt thereof in the diluted composition, prepared according to above-described method of the present invention, is about 0.5 to about 10 mg/mL, for example about 0.5 to about 7.5 mg/mL, for example about 1 to about 5 mg/mL.

In embodiments, a diluted composition of the invention, for example which is prepared from a storage stable aqueous liquid solution composition according to the invention, is for use in therapy. In embodiments, a diluted composition of the invention, for example which is prepared from a storage stable aqueous liquid solution composition according to the invention, is a pharmaceutical composition.

Dalbavancin (in particular dalbavancin hydrochloride) is indicated inter alia for the treatment bacterial infections, in particular bacterial infections caused by Gram-positive bacteria. For example, dalbavancin has antibiotic activity against Gram-positive bacteria including Staphylococcus aureus, Streptococcus pyogenes, Streptococcus agalactiae, Streptococcus anginosus, Streptococcus intermedius, and Streptococcus constellatus. Dalbavancin (in particular dalbavancin hydrochloride) demonstrates antibiotic activity against Enterococcus faecium and Enterococcus faecalis. Moreover, dalbavancin (in particular dalbavancin hydrochloride) exhibits antibiotic activity against Gram-positive bacteria of the Bacillus spp., Listeria spp., and Corynebacterium spp.

Dalbavancin (in particular dalbavancin hydrochloride) may be used to treat both methicillin-sensitive and methicillin-resistant Gram-positive bacterial infections. In particular, dalbavancin (in particular dalbavancin hydrochloride) is one of the most widely used antibiotics in the treatment of methicillin-resistant Staphylococcus aureus (MRSA).

Clinically, dalbavancin is particularly used to treat acute bacterial skin and skin structure infections (ABSSSI), in particular in adults, and in particular when said infections are caused by the aforementioned Gram-positive bacteria, including MRSA.

Accordingly, In embodiments, the present invention provides a diluted composition of the invention, for example which is prepared from a storage stable aqueous liquid solution composition according to the invention, for use in the treatment of a bacterial infection, in particular wherein the bacterial infection is caused by Gram-positive bacteria, for example wherein the Gram-positive bacteria is selected from the group consisting of Staphylococcus aureus, Staphylococcus epidermidis, Streptococcus pyogenes, Streptococcus agalactiae, Streptococcus Streptococcus anginosus, Streptococcus intermedius, Streptococcus viridans, Streptococcus constellatus, Streptococcus bovis, Enterococcus faecium, Enterococcus faecalis, Clostridium difficile, and Listeria monocytogenes. In particular, the Gram-positive bacteria is suitably selected from the group consisting of Staphylococcus aureus, Staphylococcus epidermidis, Streptococcus pyogenes, Streptococcus agalactiae, Streptococcus pneumoniae, Streptococcus anginosus, Streptococcus intermedius, Streptococcus viridans, Streptococcus constellatus, and Streptococcus bovis. For example, the bacterial infection is caused by methicillin-resistant and/or multidrug-resistant bacteria, such as a methicillin-resistant and/or multidrug-resistant Gram-positive bacteria, for example methicillin-resistant Staphylococcus aureus.

In a further embodiment, the present invention provides a diluted composition of the invention, for example which is prepared from a storage stable aqueous liquid solution composition according to the invention, for use in the treatment of acute bacterial skin and skin structure infections (ABSSSI), for example wherein said ABSSSI is caused Gram-positive bacteria.

Furthermore, In embodiments, the present invention provides use of a diluted composition of the invention, for example which is prepared from a storage stable aqueous liquid solution composition according to the invention, in the manufacture of a medicament for treating a bacterial infection, in particular wherein the bacterial infection is caused by Gram-positive bacteria. Moreover, the present invention provides use of such a diluted composition in the manufacture of a medicament for treating acute bacterial skin and skin structure infections (ABSSSI), for example wherein said ABSSSI is caused Gram-positive bacteria.

Moreover, In embodiments, the present invention provides a method of treating a bacterial infection, in particular wherein the bacterial infection is caused by Gram-positive bacteria, which comprises administering to a patient, particularly a human patient, in need thereof a therapeutically effective amount of a diluted composition described herein, which is suitably prepared from a storage stable aqueous liquid solution composition according to the invention. In addition, the present invention provides a method of treating acute bacterial skin and skin structure infections (ABSSSI), for example wherein said ABSSSI is caused Gram-positive bacteria, which comprises administering to a patient, particularly a human patient, in need thereof a therapeutically effective amount of a diluted composition described herein.

The diluted dalbavancin compositions of the present invention can be used as a sole therapy, or in combination with one or more further therapeutic agents. In embodiments, the further therapeutic agent(s) is/are antibiotics, e.g. selected from the group consisting of rifampicin, an aminoglycoside (such as gentamicin), and a beta-lactam antibiotic (such as amoxicillin). In embodiments, the present invention provides a diluted composition as described herein, which is suitably prepared from a storage stable aqueous liquid solution composition according to the invention, for use in treating a bacterial infection, in particular wherein the bacterial infection is caused by Gram-positive bacteria, with an antibiotic, e.g. selected from the group consisting of rifampicin, an aminoglycoside (such as gentamicin), and a beta-lactam antibiotic (such as amoxicillin). In embodiments, the present invention provides an antibiotic, e.g. selected from the group consisting of rifampicin, an aminoglycoside (such as gentamicin), and a beta-lactam antibiotic (such as amoxicillin) for use in treating a bacterial infection, in particular wherein the bacterial infection is caused by Gram-positive bacteria, with a diluted composition as described herein, which is suitably prepared from a storage stable aqueous liquid solution composition according to the invention.

The diluted dalbavancin compositions of the present invention may be administered (e.g. intravenously) in a therapeutically effective amount of dalbavancin or a pharmaceutically acceptable salt thereof. A therapeutically effective amount of dalbavancin or a pharmaceutically acceptable salt thereof, can be present in an amount of, for example, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg or about 3000 mg. In embodiments, the dalbavancin compositions of the present invention may be administered in a single dose of, for example, 1500 mg. In some embodiments, the dalbavancin compositions of the present invention can be administered in multiple doses, such as, for example, a 1000 mg dose followed by a 500 mg dose. If administered in multiple doses, administration is preferably at different time points, such as an initial dose of 1000 mg, then 500 mg after about 1 week.

All physical parameters referred to herein e.g. pH, pKa etc are determined at 21 °C and standard pressure.

All embodiments described above which relate to the storage stable aqueous liquid solution compositions, or diluted compositions, of the present invention may equally apply to the methods and/or uses of the invention.

### CLAUSES

The invention is further defined by the following clauses:

Clause 1. A storage stable aqueous liquid solution composition comprising:
(i) dalbavancin or a pharmaceutically acceptable salt thereof;
(ii) a neutral amino acid or a pharmaceutically acceptable salt thereof; and
(iii) a non-ionic surfactant;
wherein the pH of the composition is in the range of about 3.0 to about 7.0.

Clause 2. The storage stable aqueous liquid solution composition according to clause 1, comprising a pharmaceutically acceptable salt of dalbavancin, in particular wherein the pharmaceutically acceptable salt is selected from hydrochloride, citrate, formate, acetate, tartrate, sulphate, tosylate, nitrate, mesylate, napsylate, besylate, oxalate, maleate, phosphate, pamoate, fumarate, hippurate, succinate and carbonate.

Clause 3. The storage stable aqueous liquid solution composition according to clause 2, comprising a pharmaceutically acceptable salt of dalbavancin, in particular dalbavancin hydrochloride.

Clause 4. The storage stable aqueous liquid solution composition according to any one of clauses 1 to 3, wherein the concentration of dalbavancin or a pharmaceutically acceptable salt thereof is about 5 to about 50 mg/mL, for example about 10 to about 40 mg/ml, for example about 10 to about 30 mg/mL, or about 20 mg/ml (based on weight of dalbavancin).

Clause 5. The storage stable aqueous liquid solution composition according to any one of clauses 1 to 4, wherein the neutral amino acid or a pharmaceutically acceptable salt thereof is selected from the group consisting of glycine, methionine, proline, alanine, valine, leucine, isoleucine, phenylalanine, tyrosine, tryptophan, serine, threonine, asparagine, glutamine, and pharmaceutically acceptable salts thereof, and combinations thereof.

Clause 6. The storage stable aqueous liquid solution composition according to clause 5, wherein the neutral amino acid or a pharmaceutically acceptable salt thereof is selected from the group consisting of glycine, methionine, and proline, and pharmaceutically acceptable salts thereof, and combinations thereof.

Clause 7. The storage stable aqueous liquid solution composition according to clause 6, wherein the neutral amino acid or a pharmaceutically acceptable salt thereof is selected from the group consisting of glycine, and proline, and pharmaceutically acceptable salts thereof, and combinations thereof.

Clause 8. The storage stable aqueous liquid solution composition according to clause 7, wherein the neutral amino acid or a pharmaceutically acceptable salt thereof is glycine.

Clause 9. The storage stable aqueous liquid solution composition according to clause 7, wherein the neutral amino acid or a pharmaceutically acceptable salt thereof is proline.

Clause 10, The storage stable aqueous liquid composition according to clause 5, wherein the neutral amino acid or the pharmaceutically acceptable salt thereof is present in the form of the L isomer

Clause 11. The storage stable aqueous liquid composition according to clause 10, wherein the neutral amino acid or the pharmaceutically acceptable salt thereof is L-proline.

Clause 12. The storage stable aqueous liquid composition according to any one of clauses 1 to 11, wherein the total concentration of the neutral amino acid or the pharmaceutically acceptable salt thereof is about 50 to about 500 mM, for example about 50 to about 400 mM, for example about 100 to about 400 mM, for example about 100 to about 350 mM, for example about 100 to about 300 mM.

Clause 13. The storage stable aqueous liquid composition according to clause 12, wherein the total concentration of the neutral amino acid or the pharmaceutically acceptable salt thereof is 100-250 mM, for example 100-200 mM, such as about 100 mM or such as about 200 mM.

Clause 14. The storage stable aqueous liquid composition according to any one of clauses 1 to 13, wherein the non-ionic surfactant is selected from the group consisting of a polysorbate, an alkyl glycoside, an alkyl ether of polyethylene glycol, a block copolymer of polyethylene glycol and polypropylene glycol, and an alkylphenyl ether of polyethylene glycol, and combinations thereof.

Clause 15. The storage stable aqueous liquid composition according to clause 14, wherein the non-ionic surfactant is a polysorbate surfactant, such as polysorbate 20, polysorbate 40, polysorbate 60, or polysorbate 80.

Clause 16. The storage stable aqueous liquid composition according to clause 15, wherein the non-ionic surfactant is a polysorbate surfactant, such as polysorbate 20 or polysorbate 80.

Clause 17. The storage stable aqueous liquid composition according to clause 16, wherein the non-ionic surfactant is polysorbate 80.

Clause 18. The storage stable aqueous liquid composition according to clause 14, wherein the non-ionic surfactant is a block copolymer of polyethylene glycol and polypropylene glycol, such as poloxamer 188, poloxamer 407, poloxamer 171 and poloxamer 185.

Clause 19. The storage stable aqueous liquid composition according to clause 18, wherein the non-ionic surfactant is poloxamer 188.

Clause 20. The storage stable aqueous liquid composition according to any one of clauses 1 to 19, wherein the total concentration of the non-ionic surfactant is about 0.5 mg/ml or more, for example about 1 mg/ml or more, for example about 2 mg/ml or more, for example about 2.5 mg/mL or more, for example about 4 mg/mL or more, for example about 5 mg/ml or more.

Clause 21. The storage stable aqueous liquid composition according to any one of clauses 1 to 20, wherein the total concentration of the non-ionic surfactant is about 30 mg/mL or less, for example about 25 mg/ml or less, for example about 20 mg/ml or less, for example about 18 mg/ml or less, for example about 16 mg/ml or less, for example about 15 mg/ml or less.

Clause 22. The storage stable aqueous liquid composition according to any one of clauses 1 to 21, wherein the total concentration of the non-ionic surfactant is about 0.5 to about 30 mg/mL, for example about 0.5 to about 25 mg/mL, for example about 1 to about 20 mg/mL, for example about 2 to about 20 mg/mL, for example about 5 to about 20 mg/mL or about 2 to about 18 mg/mL, for example about 5 to about 15 mg/mL, or about 2 to about 15 mg/mL.

Clause 23. The storage stable aqueous liquid composition according to any one of clauses 1 to 22, wherein the pH of the composition is in the range about 3.5 to about 7.0, for example about 3.5 to about 6.5, for example about 3.5 to about 6.0, for example about 4.0 to about 6.0, for example about 4.0 to about 5.5, for example about 4.0 to about 5.0, or about 5.0 to about 5.5, or about 4.5, or about 5.0, or about 5.5.

Clause 24. The storage stable aqueous liquid composition according to clause 23, wherein the pH of the composition is in the range of about 4.5 to about 5.5, such as about 5.0.

Clause 25. The storage stable aqueous liquid composition according to any one of clauses 1 to 24, further comprising an additional salt, that is any salt other than a salt which is included in the composition as a result of a specific formulation excipient, e.g. dalbavancin and/or neutral amino acid, at a total concentration of about 50 mM or less, for example about 40 mM or less, for example about 25 mM or less, for example about 10 mM or less, for example about 5 mM or less.

Clause 26. The storage stable aqueous liquid composition according to clause 25, further comprising an additional salt, that is any salt other than a salt which is included in the composition as a result of a specific formulation excipient, e.g. dalbavancin and/or neutral amino acid, at a total concentration of at a total concentration of 0-50 mM, for example 0-40 mM, for example 0-30 mM, for example 0-20 mM, for example 0-10 mM, for example 0-5 mM.

Clause 27. The storage stable aqueous liquid composition according to clause 25 or clause 26, wherein the composition is substantially free of, e.g. is free of, an additional salt, that is any salt other than a salt which is included in the composition as a result of a specific formulation excipient, e.g. dalbavancin and/or neutral amino acid.

Clause 28. The storage stable aqueous liquid composition according to any one of clauses 25 to 27, wherein the additional salt is sodium chloride, sodium lactate, or combinations thereof.

Clause 29. The storage stable aqueous liquid composition according to any one of clauses 1 to 28, wherein the composition is substantially free of, e.g. is free of, N-acetyl-D-alanine and/or N-acetyl-glycine and/or N-acetyl-methionine.

Clause 30. The storage stable aqueous liquid composition according to clause 29, wherein the composition is substantially free of, e.g. is free of, N-acetyl-D-alanine and N-acetyl-glycine and N-acetyl-methionine.

Clause 31. The storage stable aqueous liquid composition according to any one of clauses 1 to 30, wherein the total ionic strength of the composition, excluding the contribution of dalbavancin or the pharmaceutically acceptable salt thereof, is suitably less than 100 mM, for example less than 80 mM, for example less than 75 mM, for example less than 60 mM, for example less than 50 mM, for example less than 40 mM, less than 35 mM, less than 30 mM, less than 25 mM or less than 20 mM.

Clause 32. The storage stable aqueous liquid composition according to any one of clauses 1 to 31, wherein the composition further comprises a buffer.

Clause 33. The storage stable aqueous liquid composition according to clause 32, wherein the buffer is selected from the group consisting of maleate, sulphite, aspartame, aspartate, glutamate, tartrate, gluconate, adenine, succinate, ascorbate, benzoate, phenylacetate, gallate, cytosine, p-aminobenzoic acid, sorbate, acetate, propionate, alginate, urate, 2-(N-morpholino) ethanesulphonic acid, bicarbonate, bis(2-hydroxyethyl) iminotris(hydroxymethyl) methane, N-(2-acetamido)-2-iminodiacetic acid, 2-[(2-amino-2-oxoethyl)amino]ethanesulphonic acid, piperazine-N,N′-bis(2-ethanesulphonic acid) (PIPES), phosphate, N,N-bis(2-hydroxyethyl)-2-aminoethanesulphonic acid, 3-[N,N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane, N-tris(hydroxymethyl)glycine, N-tris(hydroxymethyl)methyl-3-aminopropanesulphonic acid, citrate and salts thereof, and combinations thereof.

Clause 34. The storage stable aqueous liquid composition according to clause 33, wherein the buffer is selected from the group consisting of maleate, succinate, acetate and citrate.

Clause 35. The storage stable aqueous liquid composition according to clause 34, wherein the buffer is acetate buffer.

Clause 36. The storage stable aqueous liquid composition according to any one of clauses 32 to 35, wherein the buffer comprises ionisable groups with pKa within 2 units of the pH of the composition.

Clause 37. The storage stable aqueous liquid composition according to clause 36, wherein the buffer comprises ionisable groups with pKa within 1 unit of the pH of the composition.

Clause 38. The storage stable aqueous liquid composition according to any one of clauses 32 to 37, wherein the total concentration of the buffer is about 1 to about 20 mM, for example about 2 to about 20 mM, for example about 2 to about 18 mM, for example about 4 to about 16 mM, for example about 5 to about 15 mM, for example about 5 to about 12 mM, for example about 5 to about 10 mM.

Clause 39. The storage stable aqueous liquid solution composition according to any one of clauses 1 to 38, wherein the composition further comprises a polyol.

Clause 40. The storage stable aqueous liquid solution composition according to clause 39, wherein the polyol is selected from the group consisting of PEG300, PEG400 propylene glycol, sucrose, glycerol, trehalose, lactose, glucose, sorbitol and mannitol and combinations thereof.

Clause 41. The storage stable aqueous liquid solution composition according to clause 40, wherein the polyol is selected from the group consisting of propylene glycol, glycerol, mannitol, and glucose, and combinations thereof.

Clause 42. The storage stable aqueous liquid solution composition according to clause 41, wherein the polyol is selected from the group consisting of glucose and propylene glycol.

Clause 43. The storage stable aqueous liquid solution composition according to any one of clauses 39 to 42, wherein the total concentration of the polyol is about 10 to about 500 mM, for example about 10 to about 400 mM, for example about 20 to about 400 mM, for example about 25 to about 350 mM, for example about 50 to about 300 mM.

Clause 44. The storage stable aqueous liquid solution composition according to any one of clauses 1 to 43, wherein the composition further comprises an antioxidant.

Clause 45. The storage stable aqueous liquid solution composition according to clause 44, wherein the antioxidant is selected from the group consisting of monothioglycerol, butylated hydroxyanisole, glutathione (reduced), ascorbate, and cysteine.

Clause 46. The storage stable aqueous liquid solution composition according to clause 44 or clause 45, wherein the antioxidant is present at a concentration of about 1 to about 100 mM, for example about 2 to about 80 mM, for example about 5 to about 50 mM, for example about 10 to about 50 mM, for example about 25 to about 50 mM.

Clause 47. The storage stable aqueous liquid solution composition according to any one of clauses 1 to 46, wherein the composition further comprises a preservative, such as a phenolic or benzylic preservative.

Clause 48. The storage stable aqueous liquid solution composition according to clause 47, wherein the preservative is selected from the group consisting of phenol, m-cresol, chlorocresol, benzyl alcohol, propyl paraben and methyl paraben.

Clause 49. The storage stable aqueous liquid solution composition according to clause 48, wherein the preservative is selected from the group consisting of phenol, m-cresol and benzyl alcohol, and mixtures thereof.

Clause 50. The storage stable aqueous liquid solution composition according to any one of clauses 47 to 49, wherein the preservative is present at a concentration of 10-100 mM, for example 20-80 mM, such as 25-50 mM.

Clause 51. The storage stable aqueous liquid solution composition according to clause 1 comprising:
dalbavancin or a pharmaceutically acceptable salt thereof, at a concentration of 5-50 mg/mL, for example 10-30 mg/mL, or about 20 mg/mL;
a neutral amino acid or a pharmaceutically acceptable salt thereof, at a concentration of 50-500 mM, for example 50-400 mM, for example 100-400 mM, for example 100-300 mM;
a non-ionic surfactant, at a concentration of 1-20 mg/mL, for example 2-20 mg/mL, for example 5-20 mg/mL, for example 5-15 mg/ml;
   wherein the pH of the composition is in the range of about 3.0 to about 7.0, for example in the range of about 4.0 to about 6.0, such as about 5.0.

Clause 52. The storage stable aqueous liquid solution composition according to clause 1 comprising:
dalbavancin or a pharmaceutically acceptable salt thereof, e.g. dalbavancin hydrochloride, at a concentration of 5-50 mg/mL, for example 10-30 mg/mL, or about 20 mg/mL;
a neutral amino acid selected from the group consisting of glycine, methionine, and proline, and pharmaceutically acceptable salts thereof, and combinations thereof, at a concentration of 50-500 mM, for example 50-400 mM, for example 100-400 mM, for example 100-300 mM;
a non-ionic surfactant, which is a polysorbate surfactant, such as polysorbate 20, polysorbate 40, polysorbate 60, or polysorbate 80, and combinations thereof, at a concentration of 1-20 mg/mL, for example 2-20 mg/mL, for example 5-20 mg/mL, for example 5-15 mg/mL;
   wherein the pH of the composition is in the range of about 3.0 to about 7.0, for example in the range of about 4.0 to about 6.0, such as about 5.0.

Clause 53. The storage stable aqueous liquid solution composition according to clause 1 comprising:
dalbavancin or a pharmaceutically acceptable salt thereof, e.g. dalbavancin hydrochloride, at a concentration of 10-30 mg/mL, or about 20 mg/ml;
a neutral amino acid selected from the group consisting of glycine and proline, and pharmaceutically acceptable salts thereof, and combinations thereof, at a concentration of 100-400 mM, for example 100-300 mM;
a non-ionic surfactant, which is polysorbate 20 or polysorbate 80, or combinations thereof, at a concentration of 5-20 mg/mL, for example 5-15 mg/mL;
   wherein the pH of the composition is in the range of about 4.0 to about 6.0, such as about 5.0.

Clause 54. The storage stable aqueous liquid solution composition according to clause 1 consisting of, or consisting essentially of:
dalbavancin or a pharmaceutically acceptable salt thereof, at a concentration of 5-50 mg/mL, for example 10-30 mg/mL, or about 20 mg/mL;
a neutral amino acid or a pharmaceutically acceptable salt thereof, at a concentration of 50-500 mM, for example 50-400 mM, for example 100-400 mM, for example 100-300 mM;
a non-ionic surfactant, at a concentration of 1-20 mg/mL, for example 2-20 mg/mL, for example 5-20 mg/mL, for example 5-15 mg/ml;
water, e.g. water for injection;
optionally a buffer;
optionally a polyol;
optionally an antioxidant;
optionally a preservative;
   wherein the pH of the composition is in the range of about 3.0 to about 7.0, for example in the range of about 4.0 to about 6.0, such as about 5.0.

Clause 55. The storage stable aqueous liquid solution composition according to clause 1 consisting of, or consisting essentially of:
dalbavancin or a pharmaceutically acceptable salt thereof, e.g. dalbavancin hydrochloride, at a concentration of 5-50 mg/mL, for example 10-30 mg/mL, or about 20 mg/mL;
a neutral amino acid selected from the group consisting of glycine, methionine, and proline, and pharmaceutically acceptable salts thereof, and combinations thereof, at a concentration of 50-500 mM, for example 50-400 mM, for example 100-400 mM, for example 100-300 mM;
a non-ionic surfactant, which is a polysorbate surfactant, such as polysorbate 20, polysorbate 40, polysorbate 60, or polysorbate 80, and combinations thereof, at a concentration of 1-20 mg/mL, for example 2-20 mg/mL, for example 5-20 mg/mL, for example 5-15 mg/ml;
water, e.g. water for injection;
optionally a buffer;
optionally a polyol;
optionally an antioxidant;
optionally a preservative;
   wherein the pH of the composition is in the range of about 3.0 to about 7.0, for example in the range of about 4.0 to about 6.0, such as about 5.0.

Clause 56. The storage stable aqueous liquid solution composition according to clause 1 consisting of, or consisting essentially of:
dalbavancin or a pharmaceutically acceptable salt thereof, e.g. dalbavancin hydrochloride, at a concentration of 10-30 mg/mL, or about 20 mg/mL;
a neutral amino acid selected from the group consisting of glycine and proline, and pharmaceutically acceptable salts thereof, and combinations thereof, at a concentration of 100-400 mM, for example 100-300 mM;
a non-ionic surfactant, which is polysorbate 20 or polysorbate 80, or combinations thereof, at a concentration of 5-20 mg/mL, for example 5-15 mg/ml;
water, e.g. water for injection;
optionally a buffer;
optionally a polyol;
optionally an antioxidant;
optionally a preservative;
   wherein the pH of the composition is in the range of about 4.0 to about 6.0, such as about 5.0.

Clause 57. A diluted form of the storage stable aqueous liquid solution composition according to any one of clauses 1 to 56, wherein the storage stable aqueous liquid solution composition is diluted with an aqueous diluent, for example selected from 0.9% saline, 5% glucose in water, and water (e.g. sterile water for injection or bacteriostatic water for injection).

Clause 58. The diluted form of the storage stable aqueous liquid solution composition according to clause 57, wherein the concentration of dalbavancin or pharmaceutically acceptable salt thereof in the diluted composition is about 2 to about 25%, for example about 5 to about 15%, such as about 10% of the concentration in the storage stable aqueous liquid solution composition prior to dilution.

Clause 59. The diluted form of the storage stable aqueous liquid solution composition according to clause 57 or clause 58, wherein the concentration of dalbavancin or pharmaceutically acceptable salt thereof in the diluted composition is about 0.5 to about 10 mg/mL, for example about 0.5 to about 7.5 mg/mL, for example about 1 to about 5 mg/mL.

Clause 60. The diluted form of the storage stable aqueous liquid solution composition according to any one of clauses 57 to 59, wherein the diluted composition is a pharmaceutical composition.

Clause 61. The diluted form of the storage stable aqueous liquid solution composition according to any one of clauses 57 to 60, wherein the diluted composition is for use in therapy.

Clause 62. The diluted form of the storage stable aqueous liquid solution composition for use according to clause 61, for use in the treatment of a bacterial infection.

Clause 63. The diluted form of the storage stable aqueous liquid solution composition for use according to clause 62, wherein the bacterial infection is caused by Gram-positive bacteria, in particular wherein the Gram-positive bacteria is selected from the group consisting of Staphylococcus aureus, Staphylococcus epidermidis, Streptococcus pyogenes, Streptococcus agalactiae, Streptococcus pneumoniae, Streptococcus anginosus, Streptococcus intermedius, Streptococcus viridans, Streptococcus constellatus, Streptococcus bovis, Enterococcus faecium, Enterococcus faecalis, Clostridium difficile, and Listeria monocytogenes.

Clause 64. The diluted form of the storage stable aqueous liquid solution composition for use according to clause 63, wherein the bacterial infection is caused Gram-positive bacteria selected from the group consisting of Staphylococcus aureus, Staphylococcus epidermidis, Streptococcus pyogenes, Streptococcus agalactiae, Streptococcus pneumoniae, Streptococcus anginosus, Streptococcus intermedius, Streptococcus viridans, Streptococcus constellatus, and Streptococcus bovis.

Clause 65. The diluted form of the storage stable aqueous liquid solution composition for use according to any one of clauses 62 to 64, wherein the bacterial infection is caused by methicillin-resistant and/or multidrug-resistant bacteria.

Clause 66. The diluted form of the storage stable aqueous liquid solution composition for use according to clause 65, wherein the bacterial infection is caused by methicillin-resistant Staphylococcus aureus (MRSA).

Clause 67. The diluted form of the storage stable aqueous liquid solution composition for use according to clause 61, for use in the treatment of acute bacterial skin and skin structure infections (ABSSSI), in particular wherein said ABSSSI is caused Gram-positive bacteria.

Clause 68. A method of treating a bacterial infection, in particular wherein said bacterial infection is caused by Gram-positive bacteria, which comprises administering to a patient in need thereof a therapeutically effective amount of a diluted form of the storage stable aqueous liquid solution composition according to any one of clauses 57 to 60.

Clause 69. Use of a diluted form of the storage stable aqueous liquid solution composition according to any one of clauses 57 to 60, in the manufacture of a medicament for treating a bacterial infection, in particular wherein said bacterial infection is caused by Gram-positive bacteria.

Clause 70. The diluted form of the storage stable aqueous liquid solution composition for use, method, or use, according to any one of clauses 61 to 69, for use in combination with one or more other therapeutic agents.

Clause 71. The diluted form of the storage stable aqueous liquid solution composition for use, method, or use, according to clause 70, wherein the further therapeutic agent(s) is/are antibiotics, e.g. selected from the group consisting of rifampicin, an aminoglycoside (such as gentamicin), and a beta-lactam antibiotic (such as amoxicillin).

Clause 72. A method of preparing a solution for intravenous administration by diluting a storage stable aqueous liquid solution composition according to any one of clauses 1 to 56, with an aqueous diluent, for example selected from 0.9% saline, 5% glucose in water, and water (e.g. sterile water for injection or bacteriostatic water for injection).

Clause 73. The method according to clause 72, wherein the concentration of dalbavancin or pharmaceutically acceptable salt thereof in the diluted composition is about 2 to about 25%, for example about 5 to about 15%, such as about 10% of the concentration in the storage stable aqueous liquid solution composition prior to dilution.

Clause 74. The method according to clause 72 or clause 73, wherein the concentration of dalbavancin or a pharmaceutically acceptable salt thereof in the diluted composition is about 0.5 to about 10 mg/mL, for example about 0.5 to about 7.5 mg/mL, for example about 1 to about 5 mg/mL.

Clause 75. A vial, which is suitably a glass vial, containing the storage stable aqueous liquid solution composition according to any one of clauses 1 to 56.

Clause 76. The vial according to clause 75, which is a multiple dose vial.

Clause 77. An intravenous (IV) solution bag, which is suitably constructed from plastic such as PVC, polyolefin, polypropylene, a blend of PO and PP (polyolefin blend), polyurethane, or a material that is substantially free of PVC, plasticizers, adhesives and latex, such as DEHP (di(2-ethylhexyl) phthalate), containing the diluted composition according to any one of clauses 57 to 60.

Clause 78. An injection device containing the diluted composition according to any one of clauses 57 to 60.

### EXAMPLES

### General Methods

### Reversed-Phase Chromatography Method (RP-HPLC)

High performance reverse phase chromatography is performed using a C18 column and a UV detector (280 nm). The sample comprising dalbavancin (or pharmaceutically acceptable salt thereof) is injected into Mobile Phase A and subsequently eluted using a gradient of Mobile Phase A and Mobile Phase B. Mobile Phase A is a mixture of 25 mM sodium dihydrogen phosphate buffer and acetonitrile in a volume per volume ratio of 86:14. Mobile Phase B is a mixture of 25 mM sodium dihydrogen phosphate buffer and acetonitrile in a volume per volume ratio of 72:28.

### Visual Assessment Method

Visible particles are suitably detected using the 2.9.20. European Pharmacopoeia Monograph (Particulate Contamination: Visible Particles). The apparatus required consists of a viewing station comprising:
a matt black panel of appropriate size held in a vertical position
a non-glare white panel of appropriate size held in a vertical position next to the black panel
an adjustable lampholder fitted with a suitable, shaded, white-light source and with a suitable light diffuser (a viewing illuminator containing two 13 W fluorescent tubes, each 525 mm in length, is suitable). The intensity of illumination at the viewing point is maintained between 2000 lux and 3750 lux.

Any adherent labels are removed from the container and the outside washed and dried. The container is gently swirled or inverted, ensuring that air bubbles are not introduced, and observed for about 5 s in front of the white panel. The procedure is repeated in front of the black panel. The presence of any particles and opalescence is recorded. The visual scores are ranked as follows:
Visual score: Clear Solution (no opalescence and virtually free of particles)
Visual score: Slightly Opalescent
Visual score: Opalescent

### High Accuracy Subvisible Particle Analysis (HIAC) Method

HIAC is performed via Light Obscuration using a Beckman-Coulter HIAC 9703. The sample comprising dalbavancin (or pharmaceutically acceptable salt thereof) at 20 mg/ml is subjected to light obscuration to determine particle size. As per US Pharmacopeia <787> the number of test aliquots was 4 (1^{st} run discarded, average taken from final 3 runs). The particle size threshold tested in the Examples below is ≥10 μm. Results are expressed as number of particles ≥10 μm per 25 mL container.

### Multi-Angle Dynamic Light Scattering (MADLS) Method

Multi-Angle Dynamic Light Scattering (MADLS) was performed using a Malvern Zetasizer Ultra Red (ZSU3305). MADLS uses three different angles (back 173°, side 90° and forward 13° scattering detection) and combines the information obtained into one size distribution plot. The sample comprising dalbavancin (or pharmaceutically acceptable salt thereof) is placed in a ZEN2112 cuvette and subjected to MADLS at 20 mg/mL. Results are expressed as the wavelength (nm) of the highest intensity peak observed.

### Example 1: Effect of pH on the Stability of Dalbavancin Compositions

The effect of pH on the stability of dalbavancin compositions following storage for 4 weeks at 40 °C was firstly investigated using the RP-HPLC method described in General Methods. All compositions contained 20 mg/mL dalbavancin (as hydrochloride), 5 mM acetate buffer, and 300 mM mannitol. The results are shown in Table 1 which shows the increase in % impurities observed in the compositions following storage for 4 weeks at 40 °C.

TABLE 1
Stability of dalbavancin (as hydrochloride, 20
mg/mL) compositions assessed using RP-HPLC.

| | | Increase in total impurities |
|---|---|---|
| Formulation | pH | (4 weeks at 40 °C) |
| F1 | 3.0 | 16.2 |
| F2 | 4.0 | 15.6 |
| F3 | 4.5 | 9.4 |
| F4 | 5.0 | 8.0 |
| F5 | 5.5 | 7.1 |

It can be seen from Table 1 that the pH range 4.5-5.5 was the most stable, with the degradation rate accelerating at pH values below this range. In particular, the formulations were most stable at pH values of 5.0 and 5.5. It was also observed that preparing dalbavancin hydrochloride formulations at pH 5.5 was more challenging than at pH 5.0 due to reduced solubility of the dalbavancin hydrochloride.

The effect of pH on the stability of dalbavancin compositions following storage for 4 weeks at 40 °C was further investigated in the presence of additional excipients, using the RP-HPLC method described in General Methods. Here, all compositions containing 20 mg/ml dalbavancin (as hydrochloride), 5 mM acetate buffer, 300 mM propylene glycol, and 100 mM proline. The results are shown in Table 2 which shows the increase in % impurities observed in the compositions following storage for 4 weeks at 40 °C.

TABLE 2
Stability of dalbavancin hydrochloride (20
mg/mL) compositions assessed using RP-HPLC.

| | | Increase in total impurities |
|---|---|---|
| Formulation | pH | (4 weeks at 40 °C) |
| F7 | 4.0 | 4.9 |
| F8 | 4.5 | 4.3 |
| F9 | 5.0 | 3.7 |
| F10 | 5.5 | 3.7 |

It can be further seen from Table 2 that the pH values of 5.0 and 5.5 resulted in the most stable dalbavancin compositions, even in the presence of the above-described additional excipients. Due to the above-described difficulties in solubilising dalbavancin hydrochloride at pH 5.5, it may be considered that a pH of 5.0 is optimal, although the pH values of 5.5, 4.5, and even 4.0 may also be suitable in certain conditions.

### Example 2: Effect of a Neutral Amino Acid on the Stability of Dalbavancin Compositions

The effect of a neutral amino acid, and in particular L-proline, on the stability of dalbavancin compositions following storage for 4 weeks at 40 °C was firstly investigated using the RP-HPLC method described in General Methods. All compositions contained 20 mg/ml dalbavancin (as hydrochloride), 5 mM acetate buffer, and 300 mM mannitol, and were adjusted to pH 4.0. The results are shown in Table 3 which shows the increase in % impurities observed in the compositions following storage for 4 weeks at 40 °C.

TABLE 3
Stability of dalbavancin (as hydrochloride, 20
mg/mL) compositions assessed using RP-HPLC.

| | L-proline | Increase in total impurities |
|---|---|---|
| Formulation | (mM) | (4 weeks at 40 °C) |
| F11 | 0 | 15.6 |
| F12 | 100 | 9.2 |

It can be seen from Table 3 that the presence of L-proline, a neutral amino acid, at a concentration of 100 mM, provides a dalbavancin composition with significantly improved stability, as represented by a significantly decreased rate of impurity formation.

The effect of a neutral amino acid, and in particular L-proline, on the stability of dalbavancin compositions following storage for 4 weeks at 40 °C was further investigated using the RP-HPLC method described in General Methods, when the dalbavancin compositions in question were adjusted to pH 5.0. All compositions contained 20 mg/mL dalbavancin hydrochloride, 5 mM acetate buffer, and 300 mM propylene glycol, and were adjusted to pH 5.0. The results are shown in Table 4 which shows the increase in % impurities observed in the compositions following storage for 4 weeks at 40 °C.

TABLE 4
Stability of dalbavancin (as hydrochloride, 20
mg/mL) compositions assessed using RP-HPLC.

| | L-proline | Increase in total impurities |
|---|---|---|
| Formulation | (mM) | (4 weeks at 40 °C) |
| F13 | 0 | 6.9 |
| F14 | 100 | 6.3 |
| F15 | 200 | 5.5 |

It can be seen from Table 4 that the presence of L-proline, a neutral amino acid, provides a dalbavancin composition with significantly improved stability, as represented by a significantly decreased rate of impurity formation. Moreover, L-proline is able to improve the stability of dalbavancin compositions in a concentration-dependent manner. In particular, the presence of L-proline at a concentration of 200 mM results in a more significant improvement in dalbavancin stability than the presence of L-proline at a concentration of 100 mM.

### Example 3: Effect of a Non-Ionic Surfactant on the Stability of Dalbavancin Compositions

The effect of a non-ionic surfactant on the stability of dalbavancin compositions following storage for 4 weeks at 40 °C was firstly investigated using the RP-HPLC method described in General Methods. All compositions contained 20 mg/mL dalbavancin (as hydrochloride), 5 mM acetate buffer, and 300 mM propylene glycol, and were adjusted to pH 4.0. The results are shown in Table 5 which shows the increase in % impurities observed in the compositions following storage for 4 weeks at 40 °C.

TABLE 5
Stability of dalbavancin (as hydrochloride, 20
mg/mL) compositions assessed using RP-HPLC.

| Formu- | Polysorbate 80 | \|Poloxamer 188 | Increase in total impurities |
|---|---|---|---|
| lation | (mg/mL) | (mg/mL) | (4 weeks at 40 °C) |
| F16 | 0 | — | 11.4 |
| F17 | 2 | — | 8.2 |
| F18 | 8 | — | 6.9 |
| F19 | 16 | — | 5.7 |
| F20 | — | 8 | 7.5 |

It can be seen from Table 5 that the presence of a non-ionic surfactant, which is either polysorbate 80 or poloxamer 188, results in dalbavancin compositions with significantly improved stability, as represented by a significantly decreased rate of impurity formation. Notably, each of polysorbate 80 and poloxamer 188 provides a similar increase in dalbavancin stability. Moreover, non-ionic surfactant, as represented by polysorbate 80, improve the stability of dalbavancin compositions in a concentration-dependent manner.

### Example 4: Effect of Charged Species, Such as Charged Tonicity Modifiers, on the Stability of Dalbavancin Compositions

The effect of sodium chloride, which is commonly present in pharmaceutical compositions as a charged tonicity modifier, on the stability of dalbavancin compositions following storage for 4 weeks at 40 °C was investigated using the Visual Assessment Method described in General Methods above. All compositions contained 20 mg/mL dalbavancin (as hydrochloride), and 5 mM acetate buffer, and were adjusted to pH 4.0. The results are shown in Table 6 which shows the Visual Assessment Score of dalbavancin compositions following storage for 4 weeks at 40 °C.

TABLE 6
Stability of dalbavancin (as hydrochloride, 20 mg/mL) compositions
assessed using the Visual Assessment Method.

| | Sodium chloride | Visual Assessment Score |
|---|---|---|
| Formulation | (mM) | (4 weeks at 40 °C) |
| F21 | 0 | Clear Solution |
| F22 | 50 | Opalescent |

It can be seen from Table 6 that the presence of sodium chloride, in particular at a concentration of 50 mM, has detrimental impact on the stability of compositions of dalbavancin, as represented by the development of an opalescent solution, which contains particles.

### Example 5: Effect of a Polyol on the Stability of Dalbavancin Compositions

The effect of propylene glycol, which is a polyol and which is commonly present in compositions as an uncharged tonicity modifier, on the stability of dalbavancin compositions following storage for 4 weeks at 40 °C was investigated using the RP-HPLC method described in General Methods. All compositions contained 20 mg/mL dalbavancin (as hydrochloride), and 5 mM acetate buffer, and were adjusted to pH 4.0. The results are shown in Table 7 which shows the increase in % impurities observed in the compositions following storage for 4 weeks at 40 °C.

TABLE 7
Stability of dalbavancin (as hydrochloride, 20
mg/mL) compositions assessed using RP-HPLC.

| | Propylene glycol | Increase in total impurities |
|---|---|---|
| Formulation | (mM) | (4 weeks at 40 °C) |
| F23 | 0 | 12.9 |
| F24 | 50 | 12.9 |
| F25 | 300 | 11.4 |

It can be seen from Table 7 that the presence of propylene glycol, a polyol, at a concentration of 50 mM has no significant impact on the stability of dalbavancin compositions. Further, the presence of propylene glycol at a concentration of 300 mM had a beneficial impact on the stability of dalbavancin compositions, as represented by a decreased rate of impurity formation.

### Example 6: Stability of Ready-to-Dilute of Dalbavancin Compositions (20 mg/mL)

In view of the findings of the above Examples, additional stability testing of dalbavancin compositions was conducted, in particular to further investigate the effect of different combinations of excipients on the stability of dalbavancin compositions following storage for 4 weeks at 40 °C, in particular wherein said dalbavancin compositions are adjusted to pH 5.0.

Firstly, the effect of a non-ionic surfactant, in particular polysorbate 80, on the stability of dalbavancin compositions comprising 100 mM proline, following storage for 4 weeks at 40 °C was investigated using the RP-HPLC method described in General Methods. All compositions contained 20 mg/mL dalbavancin (as hydrochloride), 5 mM acetate buffer, and 100 mM L-proline, and were adjusted to pH 5.0. The results are shown in Table 8 which shows the increase in % impurities observed in the compositions following storage for 4 weeks at 40 °C.

TABLE 8
Stability of dalbavancin (as hydrochloride, 20
mg/mL) compositions assessed using RP-HPLC.

| | Polysorbate 80 | Increase in total impurities |
|---|---|---|
| Formulation | (mg/mL) | (4 weeks at 40 °C) |
| F26 | 4 | 4.5 |
| F27 | 8 | 3.9 |
| F28 | 16 | 3.2 |
| F29 | 20 | 3.0 |

It can be seen from Table 8 that the presence of polysorbate 80, a non-ionic surfactant, improves the stability of dalbavancin in a concentration-dependent manner, as represented by a decreased rate of impurity formation. Notably, this effect was observed in the presence of 100 mM proline.

Furthermore, the effect of a neutral amino acid, and in particular L-proline, on the stability of dalbavancin compositions comprising 8 mg/mL polysorbate 80, following storage for 4 weeks at 40 °C was investigated using the RP-HPLC method described in General Methods. All compositions contained 20 mg/mL dalbavancin (as hydrochloride), 5 mM acetate buffer, and 8 mg/mL polysorbate 80, and were adjusted to pH 5.0. The results are shown in Table 9 which shows the increase in % impurities observed in the compositions following storage for 4 weeks at 40 °C.

TABLE 9
Stability of dalbavancin (as hydrochloride, 20
mg/mL) compositions assessed using RP-HPLC.

| | L-proline | Increase in total impurities |
|---|---|---|
| Formulation | (mM) | (4 weeks at 40 °C) |
| F30 | 0 | 4.7 |
| F31 | 100 | 3.9 |
| F32 | 375 | 3.3 |

It can be seen from Table 9 that the presence of L-proline, a neutral amino acid, provides a dalbavancin composition with significantly improved stability, as represented by a significantly decreased rate of impurity formation, in a concentration-dependent manner. Notably, this effect was observed in the presence of 8 mg/mL polysorbate 80 which, as demonstrated above, also improves the stability of dalbavancin compositions.

In addition, the effect of different polyols, which are commonly present in pharmaceutical compositions as uncharged tonicity modifiers, on the stability of dalbavancin compositions comprising proline and polysorbate 80, which have each been demonstrated to have a beneficial effect on the stability of dalbavancin compositions, following storage for 4 weeks at 40 °C was investigated using the RP-HPLC method described in General Methods. All compositions contained 20 mg/mL dalbavancin (as hydrochloride), 5 mM acetate buffer, 8 mg/mL polysorbate 80, and 100 mM L-proline, and were adjusted to pH 5.0. The results are shown in Table 10 which shows the increase in % impurities observed in the compositions following storage for 4 weeks at 40 °C.

TABLE 10
Stability of dalbavancin (as hydrochloride, 20
mg/mL) compositions assessed using RP-HPLC.

| | Propylene | | | Increase in |
|---|---|---|---|---|
| | glycol | Glycerol | Glucose | total impurities |
| Formulation | (mM) | (mM) | (mM) | (4 weeks at 40 °C) |
| F33 | 0 | 0 | 0 | 3.9 |
| F34 | 300 | 0 | 0 | 3.7 |
| F35 | 0 | 300 | 0 | 3.9 |
| F36 | 0 | 0 | 300 | 3.6 |

It can be seen from Table 10 that the presence of a polyol, and in particular propylene glycol, glycerol, or glucose, at a concentration of 300 mM, has at worst no significant impact, and potentially a beneficial impact (see F36), on the stability of dalbavancin compositions comprising both polysorbate 80 (8 mg/mL) and L-proline (100 mM).

### Example 7: Long-Term Stability Testing of Dalbavancin Compositions

Stability of dalbavancin compositions F37 and F38 was followed for up to 4 weeks at 40 °C and 26 weeks at 2-8 °C using analytical methods described in General Methods. All compositions contained 20 mg/mL dalbavancin (as hydrochloride) and 10 mM acetate buffer and were adjusted to pH 5.0. Table 11 shows additional formulation ingredients of formulations F37 and F38 and the increase in % impurities (RP-HPLC) observed in the compositions following storage for 4 weeks at 40 °C and 26 weeks at 2-8 °C.

TABLE 11
Stability of dalbavancin (as hydrochloride, 20
mg/mL) compositions assessed using RP-HPLC.

| | | | Increase in | Increase in |
|---|---|---|---|---|
| | Polysorbate | | total impurities | total impurities |
| Formu- | 80 | Proline | (4 weeks | (26 weeks |
| lation | (mg/mL) | (mM) | at 40 °C) | at 2-8 °C) |
| F37 | 10 | 225 | 3.6 | 0.04 |
| F38 | 20 | 225 | 3.1 | 0.04 |

Stability of compositions F37 and F38 is presented in more detail in Table 12, using additional analytical methods. To provide the most comprehensive summary of dalbavancin stability the results are presented as absolute values at each time-point in Table 12.

TABLE 12
Stability of dalbavancin (as hydrochloride, 20 mg/mL) compositions
assessed using RP-HPLC, Visual Analysis, MADLS and HIAC.

| | | | 4 | 8 | 12 | 26 |
|---|---|---|---|---|---|---|
| Formu- | | 0 | weeks | weeks | weeks | weeks |
| lation | Method | weeks | (40 °C) | (2-8 °C) | (2-8 °C) | (2-8 °C) |
| F37 | RP-Total - | 3.06 | 6.63 | 3.16 | 3.16 | 3.10 |
| | Impurities (%) | | | | | |
| | Visual | Clear | Clear | Clear | Clear | Clear |
| | Assessment | Solution | Solution | Solution | Solution | Solution |
| | MADLS (nm)* | 4.75 | 5.01 | 5.25 | 5.08 | 5.52 |
| | HIAC ** | 433 | 208 | 608 | 1508 | 441 |
| F38 | RP-Total | 3.05 | 6.11 | 3.16 | 3.17 | 3.09 |
| | Impurities | | | | | |
| | Visual | Clear | Clear | Clear | Clear | Clear |
| | Assessment | Solution | Solution | Solution | Solution | Solution |
| | MADLS (nm)* | 5.09 | 5.54 | 6.26 | 5.63 | 5.92 |
| | HIAC ** | 316 | 225 | 42 | 1491 | 391 |

*Main peak particle size (nm)
** Particle count >10 μm per container volume (25 mL) - the US Pharmacopeia limit is <6000 particles per container.

Formulations F37 and F38 have very good stability in terms of total impurities (assessed by RP-HPLC), particle size (assessed by DLS) and very low count of subvisible particles. The results also confirm higher concentration of polysorbate 80 (20 mg/mL) results in a slightly better stability than a lower concentration of polysorbate 80 (10 mg/mL), particularly in terms of impurity formation at 40 °C (assessed by RP-HPLC). However, at 2-8 °C the effect of polysorbate concentration appears minimal.

### Example 8: Effect of N-Acetyl-D-Alanine on the Solubility of Dalbavancin Compositions

The effect of N-acetyl-D-alanine on stability of dalbavancin was studied in the presence of mannitol (i.e. a polyol). Formulations F39 and F40 contained 20 mg/ml dalbavancin (as hydrochloride) and 5 mM acetate buffer and were adjusted to pH 4.0. The results are shown in Table 13 which shows additional formulation ingredients of formulations F39 and F40 and the visual observation following the preparation of the formulations. As shown in Table 13 the presence of N-acetyl-D-alanine resulted in immediate opalescence and dalbavancin precipitation. N-acetyl-D-alanine is thus detrimental to dalbavancin stability.

TABLE 13
Stability of dalbavancin (as hydrochloride, 20 mg/mL) compositions
assessed using the Visual Assessment Method.

| | | N-Acetyl-D- | Visual Assessment Score |
|---|---|---|---|
| Formulation | Mannitol (mM) | Alanine (mM) | following preparation |
| F39 | 300 | 50 | Opalescent* |
| F40 | 300 | — | Clear solution |

*Precipitation was observed

### Example 9: Effect of Lactic Acid/Sodium Lactate on Stability of Dalbavancin Compositions

The effect of D/L-lactic acid on stability of dalbavancin was studied in the presence of mannitol (i.e. a polyol). Formulations F41 and F42 contained 20 mg/mL dalbavancin (as hydrochloride) and 5 mM acetate buffer and were adjusted to pH 4.0. D/L-lactic acid was added to the desired concentration. Due to the pH adjustment (pH 4.0) and the pKa of lactic acid (3.7) more than 50% of this additive will be present in the form of sodium D/L-lactate. The results are shown in Table 14 which shows additional formulation ingredients of formulations F41 and F42 as well as the increase in total impurities after 4 weeks at 40 °C following the preparation of the formulations. As shown in Table 14 the presence of D/L-lactic acid (mostly converted to sodium D/L-lactate due to pH adjustment) resulted in a more rapid increase in total impurities and is thus detrimental to dalbavancin stability.

TABLE 14
Stability of dalbavancin (as hydrochloride, 20
mg/mL) compositions assessed using RP-HPLC.

| Formu- | | D/L Lactic | Increase in total |
|---|---|---|---|
| lation | Mannitol (mM) | Acid (mM) | impurities (4 weeks at 40 °C) |
| F41 | 300 | — | 15.6 |
| F42 | 300 | 50 | 18.1 |

### SUMMARY

The results of the above Examples demonstrate that a dalbavancin composition with improved stability is provided by the presence of (i) a neutral amino acid, such as L-proline, and (ii) a non-ionic surfactant, such as a polysorbate or poloxamer surfactant.

It should be noted that dalbavancin compositions comprising both of these excipients have improved stability relative to a compositions comprising either a neutral amino acid, such as L-proline, or a non-ionic surfactant, such as a polysorbate or poloxamer surfactant, alone. For example, the formulations F27 and F31 of Example 6, which comprise 100 mM L-proline and 8 mg/mL polysorbate 80, have improved stability, as represented by a decreased rate of impurity formation, relative to F12 of Example 2, which comprises 100 mM L-proline but not 8 mg/mL polysorbate 80, and relative to F18 of Example 3, which comprises 8 mg/ml polysorbate 80 but not 100 mM L-proline.

Moreover, the results of the above Examples demonstrate that the presence of a polyol, such as propylene glycol, glycerol, or glucose, has at worst no significant impact and potentially a beneficial impact on the stability of dalbavancin compositions, but that the presence of a charged species, such as sodium chloride, or N-Acetyl-D-Alanine has a detrimental impact on dalbavancin stability.

### MISCELLANEOUS

All references referred to in this application, including patent and patent applications, are incorporated herein by reference to the fullest extent possible.

The application of which this description and claims form part may be used as a basis for priority in respect of any subsequent application. The claims of such subsequent application may be directed to any feature or combination of features described herein. They may take the form of product, composition, process, or use claims and may include, by way of example and without limitation, the following claims.

The invention embraces all combinations of preferred and more preferred groups and suitable and more suitable groups and embodiments of groups recited above.

What is claimed is:

1.-25. (canceled)

26. A storage stable aqueous liquid solution composition comprising:
(i) dalbavancin or a pharmaceutically acceptable salt thereof;
(ii) a neutral amino acid or a pharmaceutically acceptable salt thereof, wherein the neutral amino acid or a pharmaceutically acceptable salt thereof is selected from the group consisting of glycine, methionine, proline, alanine, valine, leucine, isoleucine, phenylalanine, tyrosine, tryptophan, serine, threonine, asparagine, glutamine, and pharmaceutically acceptable salts thereof, and combinations thereof; and
(iii) a non-ionic surfactant, wherein the non-ionic surfactant is selected from the group consisting of a polysorbate, an alkyl glycoside, an alkyl ether of polyethylene glycol, a block copolymer of polyethylene glycol and polypropylene glycol, and an alkylphenyl ether of polyethylene glycol, and combinations thereof;
wherein the pH of the composition is in the range 3.0 to 7.0.

27. The storage stable aqueous liquid solution composition according to claim 26, comprising a pharmaceutically acceptable salt of dalbavancin which is dalbavancin hydrochloride.

28. The storage stable aqueous liquid solution composition according to claim 26, wherein the concentration of dalbavancin or a pharmaceutically acceptable salt thereof is 5-50 mg/mL (based on weight of dalbavancin).

29. The storage stable aqueous liquid solution composition according to claim 28, wherein the concentration of dalbavancin or a pharmaceutically acceptable salt thereof is 20 mg/mL.

30. The storage stable aqueous liquid solution composition according to claim 26, wherein the neutral amino acid or a pharmaceutically acceptable salt thereof is selected from the group consisting of glycine, methionine, and proline, and pharmaceutically acceptable salts thereof, and combinations thereof.

31. The storage stable aqueous liquid solution composition according to claim 30, wherein the neutral amino acid or a pharmaceutically acceptable salt thereof is proline.

32. The storage stable aqueous liquid solution composition according to claim 26, wherein the total concentration of the neutral amino acid or a pharmaceutically acceptable salt thereof is 50-500 mM.

33. The storage stable aqueous liquid solution composition according to claim 26, wherein the non-ionic surfactant is a polysorbate surfactant.

34. The storage stable aqueous liquid solution composition according to claim 33, wherein the polysorbate surfactant is selected from polysorbate 20, polysorbate 40, polysorbate 60, or polysorbate 80.

35. The storage stable aqueous liquid solution composition according to claim 34, wherein the non-ionic surfactant is polysorbate 80.

36. The storage stable aqueous liquid solution composition according to claim 26, wherein the non-ionic surfactant is a block copolymer of polyethylene glycol and polypropylene glycol.

37. The storage stable aqueous liquid composition according to claim 26, wherein the total concentration of the non-ionic surfactant is 0.5-30 mg/mL.

38. The storage stable aqueous liquid composition according to claim 26, wherein the total concentration of the non-ionic surfactant is 5-15 mg/mL.

39. The storage stable aqueous liquid composition according to claim 26, wherein the pH of the composition is in the range 5.0 to 5.5.

40. The storage stable aqueous liquid composition according to claim 26, further comprising an additional salt at a total concentration of 5 mM or less.

41. The storage stable aqueous liquid composition according to claim 40, wherein the additional salt is sodium chloride, sodium lactate, or combinations thereof.

42. The storage stable aqueous liquid composition according to claim 26, wherein the composition is substantially free of N-acetyl-D-alanine and/or N-acetyl-glycine and/or N-acetyl-methionine.

43. The storage stable aqueous liquid composition claim 26, wherein the composition further comprises a buffer.

44. The storage stable aqueous liquid composition according to claim 43, wherein the buffer is acetate buffer.

45. The storage stable aqueous liquid composition according to claim 43, wherein the total concentration of the buffer is 1-20 mM.

46. The storage stable aqueous liquid solution composition according to claim 26, wherein the composition further comprises a polyol.

47. The storage stable aqueous liquid solution composition according to claim 46, wherein the polyol is selected from the group consisting of propylene glycol, glycerol, mannitol, and glucose, and combinations thereof.

48. The storage stable aqueous liquid solution composition according to claim 26, wherein the composition further comprises an antioxidant.

49. The storage stable aqueous liquid solution composition according to claim 26, wherein the composition further comprises a preservative.

50. The storage stable aqueous liquid solution composition according to claim 26, further comprising an aqueous diluent selected from 0.9% saline, 5% glucose in water, and water.

51. The storage stable aqueous liquid solution composition according to claim 50, wherein the concentration of dalbavancin or pharmaceutically acceptable salt thereof is 2-25% of the concentration of dalbavancin or pharmaceutically acceptable salt thereof in the storage stable aqueous liquid composition without the aqueous diluent.

52. The storage stable aqueous liquid solution composition according to claim 50, wherein the concentration of dalbavancin or a pharmaceutically acceptable salt thereof is 0.5-10 mg/mL (based on weight of dalbavancin).
